SEQUENCE LISTING (62 sequences)

SEQ ID NO: 1 - DNA, 540 bp, Oryza sativa
  CDS (1)..(540)
atggcgtcgacggatcccatgacccgccgcttcgccgtcgcgtgcggcgtgctcagccagtacgtcaaggccaactcctcgcagccgtcgacggcggcgccggtggctcaaggtgtgagtggcctcatggccgccgccgccgccgccgccgcggcgcctgtggtccaggaacccggatgcgaggtggacggcggggggcagcagttcacgatcttctacgccgggaaggtggtggtgatcgaccgctgcacgccggccatggcggccgagctgatgcggttcgcgtcggcggcgcagggcggcggcggcgcgccagaggcgccgcccgcgctggtggacatgccgatcgcgaggaaggcgtcgctgaagcggttcctggcgaagcgcaaggccacccccgcctccgcgcggtcgtcgtacgtcgtccgtgctgcggcggcggaggaggagcagccgccggcgaagaaagcgaaggcggccgtggagaggcgcgaggattggctcgcgctgggaagccttggacacatgcactcgcgctga

SEQ ID NO: 2 - protein, 179 aa, Oryza sativa
MASTDPMTRRFAVACGVLSQYVKANSSQPSTAAPVAQGVSGLMAAAAAAAAAPVVQEPGCEVDGGGQQFTIFYAGKVVVIDRCTPAMAAELMRFASAAQGGGGAPEAPPALVDMPIARKASLKRFLAKRKATPASARSSYVVRAAAAEEEQPPAKKAKAAVERREDWLALGSLGHMHSR

SEQ ID NO: 3 - DNA, 564 bp, Oryza sativa
  CDS (1)..(564)
atggcgatggaggggaagagcaggaggttcgcggtggcgtgcggggtgctcagccagtacgtgagggcggagcagaagatggcggcggcggcgggggcggcaccggcgagggcggtgacgacgctgagcctgatgcctggggcggaggtggtcgtcgaggaggaggagcggagggaggttggggaggaggaggcggggccagcgacggcgccggccgcgccgctgaccatcttctacggtgggaggatggtcgtcttcgaggacttccccgcggacaaggcggcggaggtgatgcgcatggcctcctccgggatggcggcggcgccggctcagcgggagggcgccgcgctcgcggacatgcccatcatgaggaaggcgtcgctgcagcggttcttcgccaagcgcaaggaccgcctcgcggcgaccaccccctacgcccgcccgtcgccggcggagaccaaggcctccgagccggaggagaagaagacgcccacctcatggctggacctcgccgcctccgcctccgccgccgcgcgccgtgacagcctcaccatcgcgctgtga

SEQ ID NO: 4 - protein, 187 aa, Oryza sativa
MAMEGKSRRFAVACGVLSQYVRAEQKMAAAAGAAPARAVTTLSLMPGAEVVVEEEERREVGEEEAGPATAPAAPLTIFYGGRMVVFEDFPADKAAEVMRMASSGMAAAPAQREGAALADMPIMRKASLQRFFAKRKDRLAATTPYARPSPAETKASEPEEKKTPTSWLDLAASASAAARRDSLTIAL

SEQ ID NO: 5 - DNA, 630 bp, Oryza sativa
  CDS (1)..(630)
atggccggtagtagcgagcagcagctggtcgccaacgcggcggcgacgacggtggccgggaacgggagcaggttcgcggtgacgtgtggcttgctcaggcagtacatgaaggagcacagtggaagcaacggcggcggtggcttcttgccggcggtgacggccatgagcctcatgaccggcggcgctgatgcggaggaggaggcgccggaggtgaggaagaccatggagctcttccctcagcaggctgggacgctgaaagacacgcaagaaaggaaggagatcaccgagaaggcgcagctgaccatcttctacggcgggagcgtggtggtgttcgacgatttccccgccgagaaggccggcgagctgatgaaactcgccggctcccgcgacagcacggcggcggcggccgtctcagacgccggcgccgcagcggggcagccttgcctaccagacatgcccatcgcgaggaaggtgtcgctgcagaggttcctggagaagaggaagaacaggatcgtcgtggcggagccactgccggagtcggagaagaaggaggcggagtcgagcaagcgtgccaagaaagacgatggcggcgcgtcgtggctccaggtgaaccccactctcagcctgtga

SEQ ID NO: 6 - protein, 209 aa, Oryza sativa
MAGSSEQQLVANAAATTVAGNGSRFAVTCGLLRQYMKEHSGSNGGGGFLPAVTAMSLMTGGADAEEEAPEVRKTMELFPQQAGTLKDTQERKEITEKAQLTIFYGGSVVVFDDFPAEKAGELMKLAGSRDSTAAAAVSDAGAAAGQPCLPDMPIARKVSLQRFLEKRKNRIVVAEPLPESEKKEAESSKRAKKDDGGASWLQVNPTLSL

SEQ ID NO: 7 - DNA, 516 bp, Oryza sativa
  CDS (1)..(516)
atggccgccgccggcagcagcagcaggttcgccgtcacgtgcggcctcctcagccagtacatgcgggagcgccagcagccgcagccgccggtgacggttcttgaggcggtggcggaggaggaggaggaggaggacgcgcggactatgcagctcttccccccgcgcgccgccgccgccgacggcgtcgcgacgccgtcggcggggacggccccgctgaccatcttctacgacgggaggatggtcgtggtcgacgacgtcccggtcgagaaggccgcggagctcatgaggctggccggctccgcgtgctcgccgccgcagccggcgcacgccgccgcgctcccggagatgccgatcgcgaggaaggcgtcgctgcagcgtttcctccagaagaggaagcacaggatcacgacaacgagcgaaccatacaagaaggcggcggtggcgtcgccggcgccggagaaaagcttcgccgtcgcgccggtgaaagatgagccggcgacatggctcgggctctga

SEQ ID NO: 8 - protein, 171 aa, Oryza sativa
MAAAGSSSRFAVTCGLLSQYMRERQQPQPPVTVLEAVAEEEEEEDARTMQLFPPRAAAADGVATPSAGTAPLTIFYDGRMVVVDDVPVEKAAELMRLAGSACSPPQPAHAAALPEMPIARKASLQRFLQKRKHRITTTSEPYKKAAVASPAPEKSFAVAPVKDEPATWLGL

SEQ ID NO: 9 - DNA, 501 bp, Oryza sativa
  CDS (1)..(501)
atggcggcggaggcggcggcgacgagcaggttcgcggcggcgtgcggcgcgctgagccagtacgtgagggcggcggataacgtccacagggcgcggacggcggcggcggcggcggcggtgcggccactcccgctcatgccgggggccgacgtcgccggagacgagcgggaggaggaaggaggcggcgccgcggcatcgtcggcggcggcgcagatgaccatcttctacggcgggcgggtgctggtgctcgacgagtgccccgcggacagggcggcggcgctgctgcggctcgccgcctcgtccaggggcgtgccccgcgacgatctcgccagcactgccgccgccgccggcgagtcggcggacctgccggtggcgaggaaggcgtcgctgcagcggttcatggagaagaggaagggcaggctcgccgcccgcggccagccgtaccggcgccacgacgccgcggcggcggcgcgcggcgaccacctcgcgctcgcgctctga

SEQ ID NO: 10 - protein, 166 aa, Oryza sativa
MAAEAAATSRFAAACGALSQYVRAADNVHRARTAAAAAAVRPLPLMPGADVAGDEREEEGGGAAASSAAAQMTIFYGGRVLVLDECPADRAAALLRLAASSRGVPRDDLASTAAAAGESADLPVARKASLQRFMEKRKGRLAARGQPYRRHDAAAAARGDHLALAL

SEQ ID NO: 11 - DNA, 993 bp, Oryza sativa
  CDS (1)..(993)
atggcagtgtcggatcatcattgcggcggcggcggccggagctggcggttcgcggtggcgtgcggcgtgctcagccggtgcgtcaaggccgaggcggcggcggcggcgaacggtcgccaccgccaccaccccacgatgctcctcatgccgggagccgacgtcgagccggacgtgagggaggaggcggcggcggcggcgcagctgaagatcatgtacggcgggcggatgctggtgttcgacgacttcttccccgcggggggcgcggtcgtcgagctggtgcgcgccgcggcgcgcgccgggcaggacgtacgacgcgcgggcgcggcacggcggcgagtcggcgacagccgtgggctcgacgctggcctgccggtggtgaggaaggtgtcgctacagcgattcgtggagaagaggcgtcgcatgcgggtataccatattttgtacactgacaagagtagtcatcatgtaccggggccgggtagatatcgatcatggcagtgtcggatcatcattgcggcggtggccggagctggcggtttcgtggtggcgtgcggcgtgctcagccggtgcgtcaaggccgaggcggcggcggcggcggcgaacggtcgccgccaccaccaccaccaccacaccacgatgctcctcatgccgggagccgacgtcgagccggacgtgagggaggaggcggctgcggcggcgcagctgaagatcatgtacggcgggcggatgctggtgttcgacgacttcttccccgcggggggcgcggtcgtcgagctggtgcgcgccgcggcgcgcgctgggcgggacgacgacggcgcgcgggcgcgacgccggcctgccggtggtgaggaaggtgtcgctgcagcggtccgtggagaagagaagtcgcaggcggctcggggcgacggcgccgtacatactcgccattcgccgccaatgctgccggcaaggacgccgggatcaggccgcactgacgacgccgcgttctactga

SEQ ID NO: 12 - protein, 330 aa, Oryza sativa
MAVSDHHCGGGGRSWRFAVACGVLSRCVKAEAAAAANGRHRHHPTMLLMPGADVEPDVREEAAAAAQLKIMYGGRMLVFDDFFPAGGAVVELVRAAARAGQDVRRAGAARRRVGDSRGLDAGLPVVRKVSLQRFVEKRRRMRVYHILYTDKSSHHVPGPGRYRSWQCRIIIAAVAGAGGFVVACGVLSRCVKAEAAAAAANGRRHHHHHHTTMLLMPGADVEPDVREEAAAAAQLKIMYGGRMLVFDDFFPAGGAVVELVRAAARAGRDDDGARARRRPAGGEEGVAAAVRGEEKSQAARGDGAVHTRHSPPMLPARTPGSGRTDDAAFY

SEQ ID NO: 13 - DNA, 609 bp, Oryza sativa
  CDS (1)..(609)
atggacgccgtcggcgcagctggcggtggcgccatgctccccgccgccgccaggcgcggccagccgccgcagccaccgtgcatgacgacggcgccggagcagcaggccgccgccggaggcgccgtgatctggccggcggcggcggcggcggaggccaaggagaagatggtcgtcgacgcccggaccatgcagctcttccccacgcgatccgcagacggcgtcgtcgtgtcgccggctccggcgccggcggcggcccaagaacgacgacggccggaagtgcatgtgacgccgtcggtgccggcgacggcgccgacggcgccattgaccatcgtctacggcggccaggtgctggtgttcgagcactacacggcggaggcggccgagaagctggtgcagcgcacgcagcatctgctggctgcggccgccggcggcggcggtgggaacaaaaacaataatgtcaccgttgtgactcccccgccggacgagccgcccatgctgctgccgccgccgcagatgccggctgcctcgggggtctccgccggcggcgtgatgccgatcgccaggaaggcgtcgctgcagcgattcctccaaaagaggaagcaaaagtga

SEQ ID NO: 14 - protein, 202 aa, Oryza sativa
MDAVGAAGGGAMLPAAARRGQPPQPPCMTTAPEQQAAAGGAVIWPAAAAAEAKEKMVVDARTMQLFPTRSADGVVVSPAPAPAAAQERRRPEVHVTPSVPATAPTAPLTIVYGGQVLVFEHYTAEAAEKLVQRTQHLLAAAAGGGGGNKNNNVTVVTPPPDEPPMLLPPPQMPAASGVSAGGVMPIARKASLQRFLQKRKQK

SEQ ID NO: 15 - DNA, 606 bp, Sorghum bicolor
  CDS (1)..(606)
atggccgccgagcagcagcagcagcctgccaaggccgccgcggccggcagcaggttcgcggtgacgtgcggcctgctgaggcagtacatgaaggagcagggcggcagcggcgccaccaggtgcctcgcgccggccgtcgccatgggcctcatgccggaagccgacgctgcggcggctgcgaccgaggagaggaccaccgtgctggagctcttcccgcagcaggctggcacgctcaaagacgagcagcaaaggaagaggaaggagcctgcggacgggagagcgccgctgaccatcttctacggcggaaagatggtggtgttcgacgacttccccgcggagaaggccgaggagctcatgcagctcgccggctccggcggcaacgccgcgcccgccgccgagaacgcgctgggacagcccagcctcacagacatgcccctcgcgaggaaggtgtcgctcaagaggttcctcgagaagaggaagaacaggctcaccgcggcggatccatacccggcggcggcaagtgagtcgtcgaccaagcagcctcctgctgttaaggaggagggcgcgccgtggctcggcgtgagctccgcgctcagcctcagctga

SEQ ID NO: 16 - protein, 201 aa, Sorghum bicolor
MAAEQQQQPAKAAAAGSRFAVTCGLLRQYMKEQGGSGATRCLAPAVAMGLMPEADAAAAATEERTTVLELFPQQAGTLKDEQQRKRKEPADGRAPLTIFYGGKMVVFDDFPAEKAEELMQLAGSGGNAAPAAENALGQPSLTDMPLARKVSLKRFLEKRKNRLTAADPYPAAASESSTKQPPAVKEEGAPWLGVSSALSLS

SEQ ID NO: 17 - DNA, 612 bp, Zea mays
  CDS (1)..(612)
atggcctccgagcatgcgcagctgtctgccaaggccgccgccggcagcaggttcgcggtgacgtacggcctgctgaggcagtacatgaaggagcagggcggcagcggcgccaccaggtgcctcgcgtcggccgtgggcgtgggcctcatgccggaaaccgacgctgctgctgcggcggcggaggcgaccgaggagaggaccaccgtgctggagctcttcccgcagcaggctggcacgctcaaagacgagcagcaaaggaagaggaaggagcgcgctgacgggagagcgccgctgaccatcttctacggcggcaagatggtggtcttcgacgacttccccgccgagaaggccgaggagctgatgcagctcgccggctccggcaacgccgcccaggacgcgctgggacagcccagcctcacagacatgcccctcgcgaggaaggtgtcgctaaagaggttcctggagaagaggaagaaccggctcaccgcggcagatccatacccagcagcggcggcgacggcggcggcaagcgagtcgtcgactaagcctccggcagtgaaagacgagggcgcgccgtgggtcggcgtgaactcggcgctcagcctcagctga

SEQ ID NO: 18 - protein, 203 aa, Zea mays
MASEHAQLSAKAAAGSRFAVTYGLLRQYMKEQGGSGATRCLASAVGVGLMPETDAAAAAAEATEERTTVLELFPQQAGTLKDEQQRKRKERADGRAPLTIFYGGKMVVFDDFPAEKAEELMQLAGSGNAAQDALGQPSLTDMPLARKVSLKRFLEKRKNRLTAADPYPAAAATAAASESSTKPPAVKDEGAPWVGVNSALSLS

SEQ ID NO: 19 - DNA, 543 bp, Zea mays
  CDS (1)..(543)
atggccatggcggcggcagcggagggcaagagccggaggttcgcgctggcctgcggggtgctgagccagtacgtgaaggccgagcagcagatggcggccgccggggcccctgccccgcgcgccccggccacgacgctgagcctcatgcccggcgcggacgtcggcgccgagcaggagccggccgccgcgagggggcaggagacggssgggtccgcctccacggcggcgccgctcaccatcttctacggcggcagggtggtcgtgttcgacgacttcccggcggagaaggcagccgaggtcatgcgcctcgcagctggcgccgagcgcccggcggccccggccccggcgccggcgccgcgcgacgacctgcctatcgcccggaaggcgtcgctgcagcggttcctggccaagcgcaaggaccgcctcgtggagcgcgccccctacgcccgcccgtccccatccgaggaggcggagaaggcgaagccggcctcctggctcgggctcggcggcacggacgccgagcgcctcaacatcgcgctgtga

SEQ ID NO: 20 - protein, 180 aa, Zea mays
  misc_feature (69)..(69): The ′Xaa′ at location 69 stands for Gly, or Ala.
MAMAAAAEGKSRRFALACGVLSQYVKAEQQMAAAGAPAPRAPATTLSLMPGADVGAEQEPAAARGQETXGSASTAAPLTIFYGGRVVVFDDFPAEKAAEVMRLAAGAERPAAPAPAPAPRDDLPIARKASLQRFLAKRKDRLVERAPYARPSPSEEAEKAKPASWLGLGGTDAERLNIAL

SEQ ID NO: 21 - DNA, 543 bp, Sorghum bicolor
  CDS (1)..(543)
atggcggccatgcccacggacagcatgactcgccgcttcgccgtcgcgtgcggcgtgctcagccagtacgtcaggaacggcgcgcccgcgacgacgatgacgccgccgccgccgttcctgaagcaggcgccggcggccggacccgccgcggcgcaggagatgatcgtcgccagcgccagcgcgccgcagcagctgaccatcttctacggcgggagggtggtggtgctggacgcgtgcccgcccgagaaggcggccgagctgatccgcctcgccgccgcggccgcgcagggagggtcgacgcagccgccggagcaggcgctggtggacatgcccatcgccaggaaggcctcgctgcggcgctttctcgccaagcgcaaggacaggtcctcttccgcctccccggcgccctacgacgaccgtcaggacgacgacgagccgcccgcgcccaagaagggcaagatggcggcggcggcgacgcgcgaggagccttcctcctcttcttggctcgcgctcggcagcctgtgctccatgcacagccgttga

SEQ ID NO: 22 - protein, 180 aa, Sorghum bicolor
MAAMPTDSMTRRFAVACGVLSQYVRNGAPATTMTPPPPFLKQAPAAGPAAAQEMIVASASAPQQLTIFYGGRVVVLDACPPEKAAELIRLAAAAAQGGSTQPPEQALVDMPIARKASLRRFLAKRKDRSSSASPAPYDDRQDDDEPPAPKKGKMAAAATREEPSSSSWLALGSLCSMHSR

SEQ ID NO: 23 - DNA, 714 bp, Zea mays
  CDS (1)..(714)
atggcggcgtccgcgaggcccggggagagggcgaccagcttcgccgtcgcgtgcagcctcctcagccgcttcgtccgacagaacggcgccgcgcccgcccagctaggcctcgggatcaaaggcgaggacgagcagcaaaggacgccggcgacaattaacttgctccccggagcggacggcgaggagaccgagaggaggaaggagaccatggagcttttcccgcagagcgccgggttcggtgtcaaggatcccgctgctgcccctagagagcaagaaaataaagagaagcctaaacagctcacaatcttctatggcgggaaggtgctggtgtttgacgatttccctgccgacaaggcaaaggacctaatgcagctggcgagcaagggcagccctgtggtacagaacgttgttttgcctcaaccctctgcacctgctgctgctgtcaccgacaaggccgtgccggtccccgtcatcagcttgcctgctgctcaggctgatgctaagaagcctactcgcacaaatgcctctgatatgccaattatgaggaaggcttctcttcaccgcttccttgagaagagaaaggaccgtctcaatgcaaatgcgccataccaaacttctccttcagacgctgcaccagtcaagaaggagcccgagagccaggcatggctcggattaggaccgaatgccgtcaagtccaacctgaacctgagctag

SEQ ID NO: 24 - protein, 237 aa, Zea mays
MAASARPGERATSFAVACSLLSRFVRQNGAAPAQLGLGIKGEDEQQRTPATINLLPGADGEETERRKETMELFPQSAGFGVKDPAAAPREQENKEKPKQLTIFYGGKVLVFDDFPADKAKDLMQLASKGSPVVQNVVLPQPSAPAAAVTDKAVPVPVISLPAAQADAKKPTRTNASDMPIMRKASLHRFLEKRKDRLNANAPYQTSPSDAAPVKKEPESQAWLGLGPNAVKSNLNLS

SEQ ID NO: 25 - DNA, 708 bp, Sorghum bicolor
  CDS (1)..(708)
atggcggcgtccgcgaggccaggggagagagcgaccagcttcaccgtcgcgtgcagcctcctcagccgcttcgtccgccagaacggcgccgcggctgccgagctaggcctcgggatcaaaggcgaggtcgagcagcagaggacgccggcgacaattagcttgctccccggagcggagggcgaggaggccgagaggacgaaggagaccatggagctgttcccgcagagcgccgggttcggcgtcaaggatgctgctgcccctagcgagcaagaaaataaagagaagcctaagcagctcaccatcttctatggcgggaaggtgctggtgtttgacgatttccctgccgacaaggcaaaggacctgatgcagctggccagcaagggcagccttgtggtacagaacgttgttttgcctcaaccttctgcacctgctgctgtcaccgacaaggccgtgccggccccggtcatcagcttgtctgctgctcaggctgatgctaagaagcctgctcgcacaaatgcttctgatatgcctattatgaggaaggcgtctcttcaccgcttccttgagaagagaaaggatcgtctcaatgcaaagacaccatatcagacttctccttcagatgcggcgccagtcaagaaggagcccgagagccaggcatggcttggattaggaccgaatgccgtgaagtccaacctgagcctgagctag

SEQ ID NO: 26 - protein, 235 aa, Sorghum bicolor
MAASARPGERATSFTVACSLLSRFVRQNGAAAAELGLGIKGEVEQQRTPATISLLPGAEGEEAERTKETMELFPQSAGFGVKDAAAPSEQENKEKPKQLTIFYGGKVLVFDDFPADKAKDLMQLASKGSLVVQNVVLPQPSAPAAVTDKAVPAPVISLSAAQADAKKPARTNASDMPIMRKASLHRFLEKRKDRLNAKTPYQTSPSDAAPVKKEPESQAWLGLGPNAVKSNLSLS

SEQ ID NO: 27 - DNA, 702 bp, Zea mays
  CDS (1)..(702)
atggcggcgtccgcgaggtccggggagagggcgaccagcttcgccgtcgcgtgcagcctcctcagtcgcttcgtccgccagaacggcgtcgcggctgccgagctaggcctcaggatcaaaggcgaggtcgagcagcagaggacgccggcgacaactaactcgctccccggagcggagggcgaggaggtcgaaaggaggaaggagaccatggagctgttcccgcagagcgtcgggttcagcatcaaggatgctgctgcccctagggaggagcaaggagataaagagaagcctaagcagctcacaatcttctatggcgggaaggtgctggtatttgacgatttccccgccgacaaggcaaaggatctgatgcagctggccagcaagggcagcccagtggtacagaacgttgttttgcctcaaccctctgcagttgctgctgtcactactgacaaggccgtgctggacccggtcatcagcttggccgctgctaagaagcctgctcgcacaaatgcttctgatatgcctattatgaggaaggcttctcttcaccgcttccttgagaagagaaaggaccgcctcaatgcaaagacaccatatcaaactgctccttcagatgcggcaccagtcaagaaggagccggagagccagccatggctcggattaggatcgaatgccgtggattccagcctgaacctcagctag

SEQ ID NO: 28 - protein, 233 aa, Zea mays
MAASARSGERATSFAVACSLLSRFVRQNGVAAAELGLRIKGEVEQQRTPATTNSLPGAEGEEVERRKETMELFPQSVGFSIKDAAAPREEQGDKEKPKQLTIFYGGKVLVFDDFPADKAKDLMQLASKGSPVVQNVVLPQPSAVAAVTTDKAVLDPVISLAAAKKPARTNASDMPIMRKASLHRFLEKRKDRLNAKTPYQTAPSDAAPVKKEPESQPWLGLGSNAVDSSLNLS

SEQ ID NO: 29 - DNA, 519 bp, Zea mays
  CDS (1)..(519)
atggccatggcggcagcggacggcaagagccggaggttcgcgctggcctgcggggtgctgagccagtacgtcaaggccgagcagcagcagcagcagcagatggcggccccgcgcgccccggccaccacgaccctgagcctgatgcccggcgcggacgtcggcgccgacgagcagcaggaggcggccgccgctggggccgaggagatgcccgggcccgcccccgccgcggcgccgccgctcaccatcttctacggcggcagggtggtggtgttcgaggacttcccggcggagaaggcggccgaggtcatgcgcctcgcggccggcgacgacctgcccatcgcccggaaggcgtcgctgcagcggttcctggccaagcgcaaagatcgcctcgtcgagcgcgcgccctacgcccgcccgtcgtcccccgcggaggcggtcaagccggcctcgggctcggcctcggcctcctggctcgggctccgcagcacggaagccgaccgcctcaccatcgcgctgtga

SEQ ID NO: 30 - protein, 172 aa, Zea mays
MAMAAADGKSRRFALACGVLSQYVKAEQQQQQQMAAPRAPATTTLSLMPGADVGADEQQEAAAAGAEEMPGPAPAAAPPLTIFYGGRVVVFEDFPAEKAAEVMRLAAGDDLPIARKASLQRFLAKRKDRLVERAPYARPSSPAEAVKPASGSASASWLGLRSTEADRLTIAL

SEQ ID NO: 31 - DNA, 666 bp, Sorghum bicolor
  CDS (1)..(666)
atggctggacgtgcgccggcgacggcgagggacaagaccagcttcgccgccacgtgcagcctgctgagccagtatctcaaggagaagaaggacggtggcctgcagcgccttggcggcctcgccatggcgccggcagctggagcaggagcaggaggtttccggccgccgaccaccatgaacttgctgtcagcgctcgacgcgccggccgaggagcccaccagcgacgcggcaaaggccaccgtcgaggagccaaaagaccatcacaaaagcaccgccggaaatccaagggaggcagccggagacgaggcgcaacagctgaccatcttctacggtggaaaagtggtcgtcttcgacaagttcccctccaccaaggtcaaggacttgctgcaaatcatgaaccctggcggcgaccgcgtggacagggccggtgccactgccactgtcccaacgcagagcctgcctacgccttcacacaacagtctctctgatctgccgattgcgaggaggaattcactccacaggttccttgagaagagaaaggacaggataactgcaaaggcgccataccaagtcaacagctctgctggcgttgaggcgtcgttcaaggtggagaaaccttggctgggtctgggccaagaagcggcgacagtcaagcaggagatgtga

SEQ ID NO: 32 - protein, 221 aa, Sorghum bicolor
MAGRAPATARDKTSFAATCSLLSQYLKEKKDGGLQRLGGLAMAPAAGAGAGGFRPPTTMNLLSALDAPAEEPTSDAAKATVEEPKDHHKSTAGNPREAAGDEAQQLTIFYGGKVVVFDKFPSTKVKDLLQIMNPGGDRVDRAGATATVPTQSLPTPSHNSLSDLPIARRNSLHRFLEKRKDRITAKAPYQVNSSAGVEASFKVEKPWLGLGQEAATVKQEM

SEQ ID NO: 33 - DNA, 552 bp, Zea mays
  CDS (1)..(552)
atggcgggcatgcccacggaaagcgcgacccggcgcttcgccgccgcgtgcggcgtgctcagccagtacgtccggacgaccggcgcgcccgcgatgacgccgccgccgccgttcctgaagccaccagccgccgcccaggagacgacggtcgcgccgcgcacgcagcagctgaccatcttctacggcgggagggtggtggtgctggacgcctgcccggccgacaaggcggacgagctgatccgcctcgccgcctcggcggcggccgcgcagggcccgctgcagcagccgccggaggagcaggcgctggtggacatgcccatcgcccggaaggcctcgctgcggcgcttcctcgccaagcgcaaggaccggtggtcctccgccagctctacggcctatgacgaccgccgccaggacgacgacgacgacgaggcggaggaggaggaggagccgccggcgcccaagaaaggcaagatggcggcggcggcgcgcgaggacccttcttcctcttcctggctcgcgctcggcagcatgtgctccatgcacggccgctga

SEQ ID NO: 34 - protein, 183 aa, Zea mays
MAGMPTESATRRFAAACGVLSQYVRTTGAPAMTPPPPFLKPPAAAQETTVAPRTQQLTIFYGGRVVVLDACPADKADELIRLAASAAAAQGPLQQPPEEQALVDMPIARKASLRRFLAKRKDRWSSASSTAYDDRRQDDDDDEAEEEEEPPAPKKGKMAAAAREDPSSSSWLALGSMCSMHGR

SEQ ID NO: 35 - DNA, 486 bp, Zea mays
  CDS (1)..(486)
atgacagcagcggggagcgtccagggccacggcgcgcggttcgcggcggcgtgcggcgtgctcagccggtacgtaaaagcagcagcggtggcgacgacgacgacggtggagctgcggccggcgggcacggtaggggtactccctctgatgcctggtgcggacctgtccacgcaagaggagcgcgaggcggggccggggccgtcgccgactccgagcgcgcagctgaccatctcgtacggcgggcgggtggtggtgctggacgacgtcccggcggacaaggccgccgaggtggtccggctcgcggccgcgcaaggcgcaccgcgggtgctgcgagcgccggcgaccaaggcggatgatctgcccatggcgaggaaggtgtcgctgcagcagttcatggagatgcgcaagggccgggtcgccacgcgcggttcgccctaccsccgtccggcgtmrtcgttgccggaccatctcacgctcacgctctga

SEQ ID NO: 36 - protein, 161 aa, Zea mays
  misc_feature (147)..(147): The ′Xaa′ at location 147 stands for Arg, or Pro.
MTAAGSVQGHGARFAAACGVLSRYVKAAAVATTTTVELRPAGTVGVLPLMPGADLSTQEEREAGPGPSPTPSAQLTISYGGRVVVLDDVPADKAAEVVRLAAAQGAPRVLRAPATKADDLPMARKVSLQQFMEMRKGRVATRGSPYXRPAXSLPDHLTLTL

SEQ ID NO: 37 - DNA, 807 bp, Vitis vinifera
  CDS (1)..(807)
atgtcgagctcctcggatattgcagactccggaaggtttaccgggcagagagcgccggcgaggggaccggagaagtcgagtttctcgcagacgtgcagtctgttgagccaatacataaaagagaagggcacgtttggagatctgagtctcgggatgacatgcagtctggaaggaaacggcacgcccgaatcgctccgccagacagcgacaacaacgacgatgaatttatttccaatgacggaaagatctgctggggtttccggcattcccgcccgaaatatgaatcttaagtccatgaatttgttccctcaacaagccggttttggttcctctgtttctaaggatgatgccccaaagatagttaattccagtgtaaagaagtccggaaacgtggagccccaaacagctcaaatgactattttctacggtggccaagtgatcgtgttcaatgattttccggcggacaaggcaaaggaagtcatgcgcttagctggcatgggaagctccccagtcccatctacaacagtcaaaaatccaattgatgccggcggcatggctccctccaaccccaacgttgttcctaatttcgcaaacagtttgatccaagagcgcatccaaagaccggctcaaccagttgcttgtgaactaccaattgcaaggaaagcttccctccacaggttcttggagaagagaaaagataggatcacagcaagagcaccatacaatataagcaactctcctgcaggtcctcacaaacctgctgaaagcaagtcatggctgggcttggctgccaaatctccaaagtag

SEQ ID NO: 38 - protein, 268 aa, Vitis vinifera
MSSSSDIADSGRFTGQRAPARGPEKSSFSQTCSLLSQYIKEKGTFGDLSLGMTCSLEGNGTPESLRQTATTTTMNLFPMTERSAGVSGIPARNMNLKSMNLFPQQAGFGSSVSKDDAPKIVNSSVKKSGNVEPQTAQMTIFYGGQVIVFNDFPADKAKEVMRLAGMGSSPVPSTTVKNPIDAGGMAPSNPNVVPNFANSLIQERIQRPAQPVACELPIARKASLHRFLEKRKDRITARAPYNISNSPAGPHKPAESKSWLGLAAKSPK

SEQ ID NO: 39 - DNA, 681 bp, Sorghum bicolor
  CDS (1)..(681)
atggccgcctctgggtctaggaacaacaggttcggcattacgtgcgctcgcctgcggcagttcatgatggagcagaaccacaggcagctgcggatgggtgacctcgtcgtcgggtcctcctcgttccagaggccactgcaactgaccccggtccctgtagcgacggggccggcgggttcctgggacaccggcgctacgaccttgtcgctgttccccgccggcactggcacggaggtcatcaggccagaggagaccaaggccaccctgaccatcttctacaagggacaggtggccacgttccacaacttcccggcagacagagccaaggacctcctgcagatggcaggttctgtgaccgggaaggcgccggagaaggggttcttgcagatggcagattctgtgaccgggaaggcgccggagaaaggggtgatgatgaccgccgtgccaggaaaggcggagaccagcgatgagccagcggatgcaggggcaggcatgccgcccatcgcaaggaagctgacgctacagaggttccttaggaagaggaagaacaggatcgccggcactgacgacgacggtgaccacaatgaggatgctttgccgtggaagaagagagactccgctggcaccggcaacaaccccgcggaagacgcgcctgatgatgcctcgtggctcaggctctga

SEQ ID NO: 40 - protein, 226 aa, Sorghum bicolor
MAASGSRNNRFGITCARLRQFMMEQNHRQLRMGDLVVGSSSFQRPLQLTPVPVATGPAGSWDTGATTLSLFPAGTGTEVIRPEETKATLTIFYKGQVATFHNFPADRAKDLLQMAGSVTGKAPEKGFLQMADSVTGKAPEKGVMMTAVPGKAETSDEPADAGAGMPPIARKLTLQRFLRKRKNRIAGTDDDGDHNEDALPWKKRDSAGTGNNPAEDAPDDASWLRL

SEQ ID NO: 41 - DNA, 684 bp, Zea mays
  CDS (1)..(684)
atggctggaagtgcgccggcgacggcgatggacaagaccagctttgccaccacgtgcagcctgctgagccagtacgtcaaggagaagaagggcggcctgctgcagggccttggtgccctcgccatggcgccggcagctggagaaggagctttccggccgccgaccaccatgaacttgctgtcagctctcgacgacgcgccggccgaggagcgcagcgagaaggccaccgccggggagccaaaacaccaggacaaatgcaccggcggaaatccaagggaggaggcagccggagaggaggaggaggaggcgcagcagctgaccatcttctacggcggcagagtggtcgtcttcgacatgttcccctccgccaaggtcgaggacctgctgcagatcatgagccctggcggcgacggcgtggacagggcaggcggtgccactgtccctacgcggagcctgcataggccttcacacgacagcctctctgatctgccgattgcgaggaggaattcgctccacaggtttcttgagaagagaaaggacaggataactgcaaaggcgccataccaacaagtcaacagctctgttgttggcgtcgaggcgtccaagcaggcggctggagctggggtggaaaaaccttggctggggctgggccaagaagcgacaacagcgaagctggagatgtga

SEQ ID NO: 42 - protein, 227 aa, Zea mays
MAGSAPATAMDKTSFATTCSLLSQYVKEKKGGLLQGLGALAMAPAAGEGAFRPPTTMNLLSALDDAPAEERSEKATAGEPKHQDKCTGGNPREEAAGEEEEEAQQLTIFYGGRVVVFDMFPSAKVEDLLQIMSPGGDGVDRAGGATVPTRSLHRPSHDSLSDLPIARRNSLHRFLEKRKDRITAKAPYQQVNSSVVGVEASKQAAGAGVEKPWLGLGQEATTAKLEM

SEQ ID NO: 43 - DNA, 26 bp, Artificial
  primer
ggtacccacacgcaagcttcgcagcg

SEQ ID NO: 44 - DNA, 32 bp, Artificial
  primer
gaattcgacaacagtctttccagtcttttgag

SEQ ID NO: 45 - DNA, 22 bp, Artificial
  primer
aacgagcgaaccatacaagaag

SEQ ID NO: 46 - DNA, 25 bp, Artificial
  primer
acaacagtctttccagtcttttgag

SEQ ID NO: 47 - DNA, 19 bp, Artificial
  primer
tgaaagatgagccggcgac

SEQ ID NO: 48 - DNA, 25 bp, Artificial
  primer
tccctttgtgatattctcctcctct

SEQ ID NO: 49 - DNA, 19 bp, Artificial
  primer
aaccagctgaggcccaaga

SEQ ID NO: 50 - DNA, 24 bp, Artificial
  primer
acgattgatttaaccagtccatga

SEQ ID NO: 51 - DNA, 687 bp, Oryza sativa
  CDS (1)..(687)
atggcttccgcgaaatccggggagagggggagcagcagcttcgccatggcctgcagcctgctcagccgctacgtcaggcagaacggcgcagccgccggcgagctcggcctcggcatcagaggtgaagccgatgcgaacaaggggaaggagaccatggagctgttcccccagaattccgggttcggctctgaagctgctgccgtgaaggagaccccggatgcgagggagcaagagaagcgccaattaaccatcttctacggtgggaaggtcctggtgtttgatgacttcccagctgagaaggccaaggacctgatgcagatggccagcaagagttcctccacagcgcagaactgtgtccttcttccatcttccgctactgcaaccgttgccgacaacactaaggtgtctgctgtaccagccccagcaagtgcacttcctgttgctcaggccaatgccccgaaacctgttcgtccaaatgctgctgatctgcctcaggctaggaaggcgtcgcttcaccggttcctcgagaaaagaaaggatcgccttcaggctaaagcaccctaccagggttccccttcagatgcttctccggtcaagaaggagctgcaggagagtcagccatggcttgggttaggacctcaggttgccgctcccgacctgagcttgcggcaggaatcgagccaatga

SEQ ID NO: 52 - protein, 228 aa, Oryza sativa
MASAKSGERGSSSFAMACSLLSRYVRQNGAAAGELGLGIRGEADANKGKETMELFPQNSGFGSEAAAVKETPDAREQEKRQLTIFYGGKVLVFDDFPAEKAKDLMQMASKSSSTAQNCVLLPSSATATVADNTKVSAVPAPASALPVAQANAPKPVRPNAADLPQARKASLHRFLEKRKDRLQAKAPYQGSPSDASPVKKELQESQPWLGLGPQVAAPDLSLRQESSQ

SEQ ID NO: 53 - DNA, 735 bp, Oryza sativa
  CDS (1)..(735)
atggcggcttccgcgaggcccgtcggcgtcggcggggagagggcgacgagcttcgccatggcgtgcagcctgctcagccgctacgtccgccagaacggcgccgccgccgccgagctcggcctcggcatcagaggtgagggtgaggctccgagggcggcgccggcgacgatgagcttgctgcccggggaggcggagaggaagaaggagaccatggagctcttcccgcagagcgccggctttggccagcaggatgccatcaccgccgattctgctgctgatgctagggaacaagagcctgagaagcgtcagctgaccatcttctatggtgggaaggtgctcgtgttcaacgacttcccagccgacaaggcaaagggcttgatgcagctggctagcaagggcagcccggtggctcctcagaatgccgcggcacctgcaccagcagctgttacagacaacaccaaggcccctatggccgtgccggccccggtcagtagcttgcctacagctcaggccgatgctcagaagcctgctcgcgcgaatgcttctgatatgcctattgctaggaaggcatcactccacaggttccttgagaagagaaaggatcgtcttaatgcaaagacgccataccaggcttctccttcagatgcaaccccagtcaagaaggagcctgagagccagccatggctcggactagggccgaacgccgtcgtgaagcccatagaacgcggccaatga

SEQ ID NO: 54 - protein, 244 aa, Oryza sativa
MAASARPVGVGGERATSFAMACSLLSRYVRQNGAAAAELGLGIRGEGEAPRAAPATMSLLPGEAERKKETMELFPQSAGFGQQDAITADSAADAREQEPEKRQLTIFYGGKVLVFNDFPADKAKGLMQLASKGSPVAPQNAAAPAPAAVTDNTKAPMAVPAPVSSLPTAQADAQKPARANASDMPIARKASLHRFLEKRKDRLNAKTPYQASPSDATPVKKEPESQPWLGLGPNAVVKPIERGQ

SEQ ID NO: 55 - DNA, 699 bp, Oryza sativa
  CDS (1)..(699)
atggccggccgtgcgacggcgacggcgacggcggcggggaaggacaggtccagcttcgccgtcacctgtagcctcctcagccagtttctcaaggagaagaagggcggcggcggcgggctgcaggggctcggcctcggcttgcggccggcgccggcggcgccgcccgccgcgggagctggaggtgctttccggccgccgccgaccaccatgaacttgctgtcggggctcgacgcgccggcggtggaggtggagccaaacacggcggaaacagcggccgacgagctgcctctcatcaaggccccggccgatcagcaaagcgacgaaagtgcaagtgaggcagctggagagaaggctcaacagctgaccatcttctacggtggaaaagtagtcgtctttgagaacttcccgtccaccaaggtcaaggatctcttacaaatcgtaagcacaggcgatggcgtcgacaagaacaccggcaccgcggcaacgcagagcctgccccgacccgcacacaacagtcttcccgatctgccgattgcaaggaggaactcgctccataggtttctcgagaagagaaagggcaggatgaatgcaaatgctccataccaagctaactgcaccgctgcaccgtccaagcaagctaacggtgacaaatcttggcttgggtttggccaagagatgacaataaagcaggagatatga

SEQ ID NO: 56 - protein, 232 aa, Oryza sativa
MAGRATATATAAGKDRSSFAVTCSLLSQFLKEKKGGGGGLQGLGLGLRPAPAAPPAAGAGGAFRPPPTTMNLLSGLDAPAVEVEPNTAETAADELPLIKAPADQQSDESASEAAGEKAQQLTIFYGGKVVVFENFPSTKVKDLLQIVSTGDGVDKNTGTAATQSLPRPAHNSLPDLPIARRNSLHRFLEKRKGRMNANAPYQANCTAAPSKQANGDKSWLGFGQEMTIKQEI

SEQ ID NO: 57 - DNA, 32 bp, Artificial
  synthetic DNA
tttggtaccactcagagacagacaaggacgag

SEQ ID NO: 58 - DNA, 32 bp, Artificial
  synthetic DNA
ttgactagtatgaggtttcttgggttgtactg

SEQ ID NO: 59 - DNA, 33 bp, Artificial
  synthetic DNA
tggtacctatgtgatcagcgacgtacagtacag

SEQ ID NO: 60 - DNA, 30 bp, Artificial
  synthetic DNA
tgaattccaattcagtccccataggaatcg

SEQ ID NO: 61 - DNA, 29 bp, Artificial
  synthetic DNA
gaaggtaccccgagattttctccgaacac

SEQ ID NO: 62 - DNA, 34 bp, Artificial
  synthetic DNA
ctagagctccttgtgacagataggaataatcgtg

(19) United States
(12) Patent Application Publication
(10) Pub. No.: US 2013/0305411 A1
(43) Pub. Date: Nov. 14, 2013

(54) METHOD FOR CONFERRING DISEASE RESISTANCE TO PLANT

(71) Applicants: Hideo Nakashita, Saitama (JP); Michiko Yasuda, Saitama (JP); Shuhei Shima, Saitama (JP); Satoshi Shinozaki, Tokyo (JP)

(72) Inventors: Hideo Nakashita, Saitama (JP); Michiko Yasuda, Saitama (JP); Shuhei Shima, Saitama (JP); Satoshi Shinozaki, Tokyo (JP)

(73) Assignees: MAYEKAWA MFG. CO., LTD., Tokyo (JP); RIKEN, Saitama (JP)

(21) Appl. No.: 13/878,199

(22) PCT Filed: Oct. 7, 2011
(86) PCT No.: PCT/JP2011/073259

§ 371 Date: Jul. 18, 2013

(30) Foreign Application Priority Data
Oct. 7, 2010 (JP) 2010-227734

(51) Int. Cl. C12N 15/82

(52) U.S. Cl. CPC C12N 15/8282
USPC 800/279

(57) ABSTRACT

Disease resistance is conferred to a plant by a technique completely differing from conventional techniques. The method of the present invention comprises a step of introducing at least one of the genes belonging to the TIFY family to a plant or enhancing the expression of the gene endogenous to the plant.

### TECHNICAL FIELD

The present invention relates to a method for conferring resistance to disease resulting from a pathogen such as a virus or a microorganism to a plant.

### BACKGROUND ART

Plants living in a fixed state and being unable to move from their growth sites are always subjected to various external stresses. They are thus provided with unique self defense mechanisms that differ from those of animals. Plants do not have animals' immunocompetent cells against biological stresses, which include attack by pathogens such as viruses or microorganisms. Thus, plants are thought to cause their individual cells to initiate resistance to pathogens, and then transmit signals thereof throughout the plant bodies, so as to prepare against secondary infections.

Induction of disease resistance in a plant has been achieved by processing the plant with a compound, and the results have been put into practical use. However, induction of disease resistance by processing with a compound is problematic in that it causes suppressed plant growth. For example, it has been reported in Arabidopsis thaliana that an antagonistic reciprocal inhibitory relationship is present between the action of auxin, which is a plant hormone relating to growth, and the action of salicylic acid, which is a plant hormone relating to disease resistance.

Techniques for conferring or enhancing disease resistance by genetic recombination technology have also been reported. However, suppressed growth takes place in plants with disease resistance enhanced by genetic recombination technology, which interferes with the practical use of such techniques.

Meanwhile, Non-patent Document 1 discloses that transgenic Arabidopsis thaliana caused to constitutively express a mutant AtJAZ gene (prepared by deletion or mutation of a Jas domain of an AtJAZ gene in Arabidopsis thailana) has acquired resistance to a pathogenic bacterium, Pseudomonas syringae. Specifically, Arabidopsis thaliana transformed with the mutant AtJAZ1 gene into which R205A mutation and R206A mutation have been introduced and Arabidopsis thaliana transformed with a mutant AtJAZ1 gene in which a Jas domain has been completely deleted are prepared, Resistance to the pathogenic bacterium Pseudomonas syringea in transgenic Arabidopsis thaliana is compared with that of wild-type Arabidopsis thaliana. Furthermore, Non-patent Document 1 discloses decreased interaction between a mutant AtJAZ9 prepared by introducing R223A mutation and K224A mutation and COI1. These disclosures show results such as those in Non-patent Document 1, when the JAZ protein interacts with COI1, Arabidopsis thaliana exhibits sensitivity to a pathogenic bacterium, but when the degree of interaction between the JAZ protein and COI1 decreases, Arabidopsis thaliana exhibits resistance to the pathogenic bacterium.

Moreover, Non-patent Document 2 discloses a gene group belonging to the TIFY family of rice. Genes belonging to the TIFY family encode proteins having a highly conserved TIFY motif (TIF[F/Y]XG). In addition, the JAZ gene in Arahidopsis thaliana as disclosed in Non-patent Document 1 belongs to the TIFY family since it encodes a protein having the TIFY motif and the Jas domain.

### PRIOR ART DOCUMENTS

### Non-Patent Documents

Non-patent Document 1: The Plant Journal (2008) 55, 979-988
Non-patent Document 2: Plant Mol. Biol. (2009) 71:291-305

### SUMMARY OF THE INVENTION

### Problem to be Solved by the Invention

Effective means for conferring disease resistance to plants have not yet been developed, as described above. Hence, in view of the above circumstances, an object of the present invention is to provide a method for conferring disease resistance to a plant by techniques completely differing from conventional techniques.

### Means for Solving Problem

As a result of intensive studies to achieve the above object, the present inventors have arrived at the novel finding that disease resistance can be conferred to a plant by introducing thereinto a predetermined gene belonging to the TIFY family, and thus have completed the present invention.

Specifically, the method for conferring disease resistance according to the present invention comprises a step of introducing at least one of genes belonging to the TIFY family into a plant or enhancing the expression of the gene endogenous to the plant.

Furthermore, a method for producing a plant according to the present invention comprises a step of preparing a transgenic plant by introducing at least one of the genes belonging to the TIFY family into a plant or enhancing the expression of the gene endogenous to the plant and a step of evaluating the disease resistance of a progeny plant of the transgenic plant so as to select a line with the significantly improved disease resistance.

Plants to be subjected to the present invention may be either dicotyledons or monocotyledons. In particular, plants to be subjected to the present invention are preferably monocotyledons.

A part or all of the content disclosed in the description and/or drawings of Japanese Patent Application No. 2010-227734, which is a priority document of the present application, is herein incorporated by reference.

### Effect of the Invention

According to the present invention, disease resistance can be conferred to plants and plants having improved disease resistance can be produced. Therefore, through application of the present invention, plants'own productivity can be improved, for example.

### BRIEF DESCRIPTION OF THE DRAWINGS

FIG. 1 is a characteristic diagram showing the results of spray inoculation of a line highly expressing OsTIFY11d with a blast pathogen and then measuring the number of diseased lesions that appeared on the 5^{th} leaves on day 5 after spray inoculation and photographs of the 5^{th} leaves.

FIG. 2 is a characteristic diagram showing the results of drop inoculation of the line highly expressing OsTIFY11d with a blast pathogen, measuring the progress of lesions on day 5 after inoculation, and then photographs of the leaves.

FIG. 3 shows the results of measuring the growth of the line highly expressing OsTIFY11d.

FIG. 4 is a characteristic diagram showing the results of spray inoculation of a line highly expressing OsTIFY10a, the line highly expressing OsTIFY11a, and a line highly expressing OsTIFY11e with a blast pathogen, and then measuring the number of diseased lesions that appeared on the 5^{th} leaves on day 5 after spray inoculation.

### EMBODIMENTS FOR CARRYING OUT THE INVENTION

Hereinafter, the present invention is described in detail,

In the present invention, a gene belonging to the TIFY for example, at least one gene selected from the group consisting of an OsTIFY11a gene, an OsTIFY11b gene, an OsTIFY11c gene, an OsTIFY1 id gene, an OsTIFY11e gene, an OsTIFY11f gene, an OsTIFY11g gene, an OsTIFY10a gene, an OsTIFY10b gene, and an OsTIFY10c gene, and homologous genes thereof is introduced into a plant or the expression of the gene endogenous to the plant is enhanced. Examples of techniques for enhancing the expression of such an endogenous gene include a technique for modifying the expression control region of the gene and a technique for causing compounds, plant hormones, microorganisms, and proteins such as transcription factors to act. The latter technique may involve a mechanism, by which these substances are caused to act so as to activate signals placed upstream of the above gene or may involve a completely different mechanism for enhancing the expression of the gene.

In addition, the expression of the above gene may be enhanced throughout an entire plant tissue or in at least a part of a plant tissue. The term “plant tissue” is meant to include, but is not limited to, plant organs such as leaves, stems, seeds, roots, and flowers.

Disease resistance can be improved by enhancing the expression of the above gene. Specifically, the expression of the above gene is enhanced throughout all plant tissues, so that disease resistance can be improved not only in plant tissues, but also in a whole plant. Alternatively, the expression of the above gene is enhanced in a specific plant tissue, so that disease resistance in the specific plant tissue can be improved.

In addition, the above-mentioned “expression control region” is meant to include a promoter region to which RNA polymerase binds and regions to which other transcription factors bind. An example of a technique for modifying a transcriptional control region is a technique that involves substituting a promoter region or the like among endogenous transcriptional control regions with a promoter region that enables higher-level expression.

<Gene Belonging to the TIFY Family>

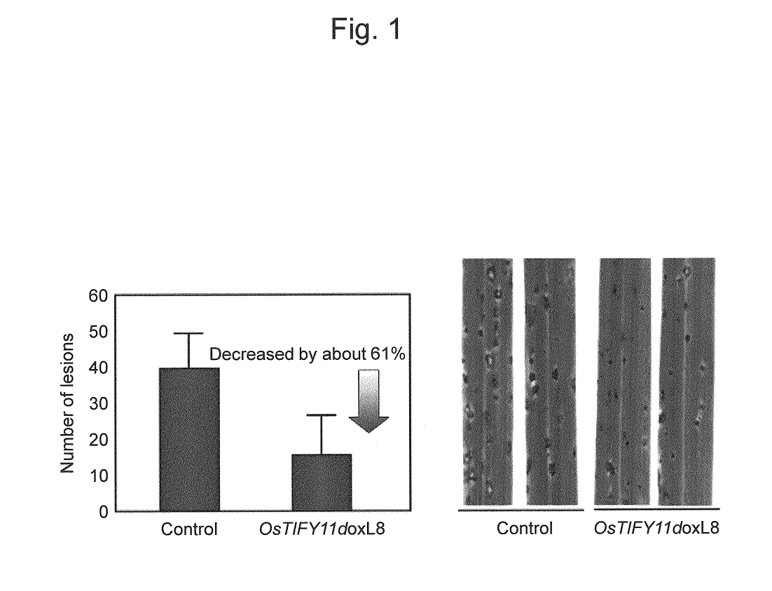

The OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene all belong to the TIFY family. As described in Reference 1 (Plant Mol. Biol. (2009) 71: 291-305, particularly FIG. 1), these genes are classified into group II of the two gene groups belonging to the rice TIFY family in the molecular phylogenetic tree thereof. In particular, the OsTIFY11a gene, the OsTIFY11b gene, the OsTIFY11c gene, the OsTIFY1 Id gene, the OsTIFY11e gene, the OsTIFY11f gene, and the OsTIFY11g gene form even a smaller group in the same molecular phylogenetic tree. Furthermore, the OsTIFY10a gene, the OsTIFY10b gene and the OsTIFY10c gene form even a further smaller group in the molecular phylogenetic tree.

It can be understood that the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene encode proteins having high sequence similarity (identity) to each other. In particular, it can be understood that the OsTIFY11a gene, the OsTIFY11b gene, the OsTIFY11c gene, the OsTIFY11d gene, the OsTIFY11e gene, the OsTIFY11f gene, and the OsTIFY11g gene encode proteins showing even higher sequence similarity (identity) to each other. It can also be understood that the OsTIFY10a gene, the OsTIFY10b gene, and the OsTIFY10c gene encode proteins showing even further higher sequence similarity (identity) to each other.

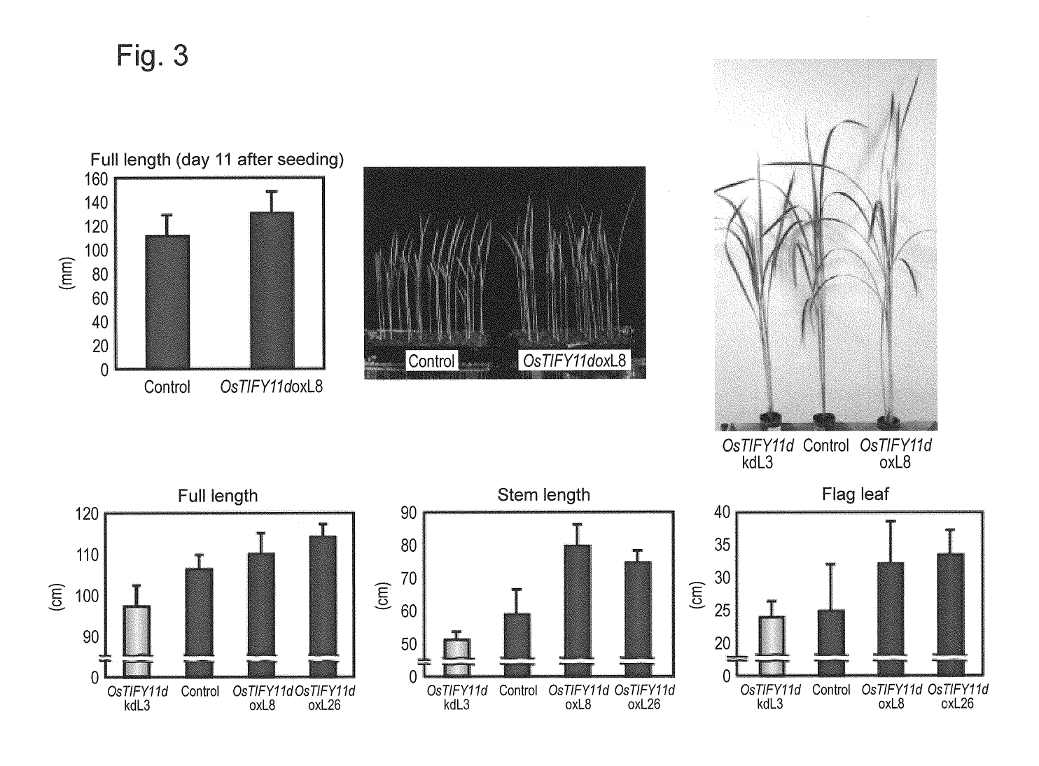

The term “a gene belonging to the TIFY family” refers to a gene encoding a protein (referred to as “TIFY protein” in the description) having a highly conserved TIFY motif (TIF[F/Y]XG). For example, TIFY motifs are seen at positions 83 to 88, 70 to 75, 73 to 78, and 73 to 78, respectively, in SEQ ID NOS: 52, 2, 8, and 10 showing the amino acid sequences of TIFY proteins, OsTIFY10a, OsTIFY11a, OsTIFY11d, and OsTIFY11e. Moreover, the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene encode proteins each having, particularly from the N terminal side, an N-terminal domain, a ZIM domain containing the TIFY motif, and a Jas domain in such an order (see FIG. 3 in Reference 1 above).

The nucleotide sequences of coding regions, amino acid sequences encoded by the nucleotide sequences, and accession Nos. in the TIGR Rice Genome Annotation Database of these genes are summarized in Table 1 below.

TABLE 1

| Gene | Nucleotide | Amino acid | |
|---|---|---|---|
| notation | sequence | sequence | TIGR Acc. No. |
| OsTIFY11a | SEQ ID NO: 1 | SEQ ID NO: 2 | Os03g08310 |
| OsTIFY11b | SEQ ID NO: 3 | SEQ ID NO: 4 | Os03g08330 |
| OsTIFY11c | SEQ ID NO: 5 | SEQ ID NO: 6 | Os03g08320 |
| OsTIFY11d | SEQ ID NO: 7 | SEQ ID NO: 8 | Os10g25290 |
| OsTIFY11e | SEQ ID NO: 9 | SEQ ID NO: 10 | Os10g25230 |
| OsTIFY11f | SEQ ID NO: 11 | SEQ ID NO: 12 | Os10g25250 |
| OsTIFY11g | SEQ ID NO: 13 | SEQ ID NO: 14 | Os03g27900 |
| OsTIFY10a | SEQ ID NO: 51 | SEQ ID NO: 52 | Os03g28940 |
| OsTIFY10b | SEQ ID NO: 53 | SEQ ID NO: 54 | Os07g42370 |
| OsTIFY10c | SEQ ID NO: 55 | SEQ ID NO: 56 | Os09g26780 |

In addition, examples of the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene are not limited to genes consisting of the nucleotide sequences and the amino acid sequences specified by the above sequence identification numbers. Specifically, the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, or the OsTIFY10c gene may be a gene encoding a protein that comprises an amino acid sequence derived from the amino acid sequence specified by one of the above sequence identification numbers by deletion, substitution, addition, or insertion of 1 or a plurality of amino acid, and functions to confer disease resistance to a plant. Here, the number of a plurality of amino acids ranges from, for example, 1 to 20, preferably 1 to 10, more preferably 1 to 7, further preferably 1 to 5, and particularly preferably 1 to 3.

In addition, deletion, substitution, or addition of amino acids can be carried out by modifying the nucleotide sequence encoding the above gene by techniques known in the art, Mutation can be introduced into a nucleotide sequence by a known technique such as the Kunkel method or the Gapped duplex method or a method in accordance therewith. For example, mutation is introduced using a kit for introducing mutation that utilizes the site-directed mutagenesis method (e.g., Mutant-K or Mutant-G (trade names) TAKARA Bio), or the kit of LA PCR in vitro Mutagenesis series (trade name, TAKARA Bio)). Also, a method for introducing mutation may be a method using chemical mutation agents represented by EMS (ethyl methanesulfonate), 5-bromouracil, 2-aminopurine, hydroxylamine, N-methyl-N′-nitro-N nitrosoguanidine, and other carcinogenic compounds, or a method using treatment with radiation rays represented by X-rays, alpha rays, beta rays, gamma rays, or ion beams, or ultraviolet treatment.

Furthermore, the OsTIFY11a gene, the OsTIFY11b gene, the OsTIFY11e gene, the OsTIFY11d gene, the OsTIFY11e gene, the OsTIFY11f gene, the OsTIFY11g gene, the OsTIFY10a gene, the OsTIFY10b gene, and the OsTIFY10c gene are not limited to genes consisting of the nucleotide sequences and the amino acid sequences specified by the above sequence identification numbers. These genes may be genes that encode proteins having amino acid sequences that have 60% or more, preferably 70% or more, more preferably 30% or more, further preferably 90% or more, and most preferably 95% or more similarity or identity to these amino acid sequences and functioning to confer disease resistance to plants. Here, the value for “similarity” or “identity” refers to a value that is found by default setting using a computer program that implements the BLAST (Basic Local Alignment Search Tool) program and a database storing gene sequence information.

As described above, a TIFY protein that is preferably used in the present invention has an N-terminal domain, a ZIM domain, and a Jas domain. For example, in the amino acids of SEQ ID NO: 52 showing the amino acid sequence of OsTIFY10a, amino acid positions 10 to 43 correspond to the N-terminal domain, amino acid positions 81 to 108 correspond to the ZIM domain, and amino acid positions 162 to 188 correspond to the Jas domain. In the amino acids of SEQ ID NO: 2 showing the amino acid sequence of OsTIFY11a, amino acid positions 7 to 29 correspond to the N-terminal domain, amino acid positions 68 to 95 correspond to the ZIM domain, and amino acid positions 113 to 139 correspond to the Jas domain. In the amino acids of SEQ ID NO: 8 showing the amino acid sequence of OsTIFY11d, amino acid positions 6 to 29 correspond to the N-terminal domain, amino acid positions 71 to 98 correspond to the ZIM domain, and amino acid positions 115 to 141 correspond to the Jas domain. In amino acids of SEQ ID NO: 10 showing the amino acid sequence of OsTIFY11e, amino acid positions 7 to 40 correspond to the N-terminal domain, amino acid positions 71 to 98 correspond to the ZIM domain, and amino acid positions 121 to 147 correspond to the Jas domain.

A TIFY protein may be a protein having an amino acid sequence that has 60% or more, preferably 70% or more, more preferably 80% or more, further preferably 90% or more, and most preferably 95% or more amino acid sequence similarity or identity to any one domain (e.g., the Jas domain) or all domains, and functioning to confer disease resistance to a plant.

Furthermore, the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene are not limited to genes consisting of the nucleotide sequences and the amino acid sequences specified by the above sequence identification numbers. Each of these genes may be a gene consisting of a polynucleotide that hybridizes under stringent conditions to at least a part of or the entire gene consisting of the nucleotide sequence and the amino acid sequence specified by the above sequence identification numbers, and, encoding a protein that functions to confer disease resistance to plants. Here, the term “stringent conditions” refers to conditions under which namely a specific hybrid is formed, but a nonspecific hybrid is not formed. For example, under such stringent conditions, a nucleic acid having high similarity or identity; that is, the complementary strand of DNA consisting of a nucleotide sequence having 60% or more, preferably 70% or more, more preferably 80% or more, further preferably 90% or more, and most preferably 95% or more similarity or identity to any one of the nucleotide sequences specified by the above sequence identification numbers hybridizes, but the complementary strand of a nucleic acid having similarity or identity lower than the above does not hybridize. More specifically, examples thereof include hybridization at 45° C. using 6×SSC (sodium chloride/sodium citrate) followed by washing at 50° C. to 65° C. with 0.2 to 1×SSC and 0.1% SDS, or hybridization at 65° C. to 70° C. with 1×SSC, followed by washing at 65° C. to 70° C. using 0.3×SSC, Hybridization is carried out by a conventionally known method, such as a method described in J. Sambrook et al. Molecular Cloning, A Laboratory Manual, 2nd Ed., Cold Spring Harbor Laboratory (1989).

Meanwhile, in the present invention, a gene homologous to any one of the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene may be introduced into a plant. Here, the term “homologous gene” refers to a gene derived from an organism other than rice and corresponds to any one of the OsTIFY11a gene, the OsTIFY11b gene, the OsTIFY11e gene, the OsTIFY11d gene, the OsTIFY11e gene, the OsTIFY11f gene, the OsTIFY11g gene, the OsTIFY10a gene, the OsTIFY10b gene, and the OsTIFY10c gene. Such homologous genes are also included in the genes belonging to the TIFY family.

The above homologous gene derived from an organism other than rice is not particularly limited and can be specified by searching a database storing gene sequences of various organisms. Specifically, for example, the DDBJ/EMBL/GenBank International Nucleotide Sequence Database or the SWISS-PROT Database are searched with the nucleotide sequences or the amino acid sequences shown in the above sequence identification numbers as query sequences, so that homologous genes can be easily searched for and identified from the known databases.

Here, the term “homologous gene” refers to a gene resulting from evolution and branching front, in general, a common ancestor gene, including a homologous gene (ortholog) of 2 types of species and a homologous gene (paralog) resulting from duplicate branching within the same species. In other words, the above term “homologous gene” is meant to include homologous genes such as an ortholog and a paralog.

In addition, a homologous gene derived from a plant other than rice can be obtained by, when the plant genome information is unknown, extracting the genome from a target plant or constructing a cDNA library of the target plant, isolating cDNA hybridizing under stringent conditions to a part of or the whole polynucleotide consisting of the above-mentioned nucleotide sequence, and then carrying out procedures according to a standard method.

More, specifically, BlastP search is carried out using the full-length amino acid sequence (SEQ ID NO: 8) of the protein encoded by the OsTIFY11d gene as a query sequence, and thus sorghum (Sorghum bicolor) and corn (Zea mays)-derived homologous genes can be searched for, Homologous genes of the OsTIFY11d gene, which are derived from Sorghum (Sorghum bicolor) and corn (Zea mays) are summarized in Table 2 below.

TABLE 2

| | | Nucleotide | Amino acid | |
|---|---|---|---|---|
| Origin | Gene notation | sequence | sequence | Identities |
| Sorghum bicolor | Sb01g045180 | SEQ ID NO: 15 | SEQ ID NO: 16 | 43% |
| Zea mays | LOC100281912 | SEQ ID NO: 17 | SEQ ID NO: 18 | 40% |
| Zea mays | LOC100283151 | SEQ ID NO: 19 | SEQ ID NO: 20 | 44% |
| Sorghum bicolor | Sb01g045190 | SEQ ID NO: 21 | SEQ ID NO: 22 | 40% |
| Zea mays | LOC100282471 | SEQ ID NO: 23 | SEQ ID NO: 24 | 36% |
| Sorghum bicolor | Sb02g039190 | SEQ ID NO: 25 | SEQ ID NO: 26 | 32% |
| Zea mays | LOC100284291 | SEQ ID NO: 27 | SEQ ID NO: 28 | 33% |
| Zea mays | LOC100284433 | SEQ ID NO: 29 | SEQ ID NO: 30 | 40% |
| Sorghum bicolor | Sb02g025720 | SEQ ID NO: 31 | SEQ ID NO: 32 | 33% |
| Zea mays | LOC100284541 | SEQ ID NO: 33 | SEQ ID NO: 34 | 35% |
| Zea mays | LOC100284979 | SEQ ID NO: 35 | SEQ ID NO: 36 | 37% |
| Vitis vinifera | LOC100254231 | SEQ ID NO: 37 | SEQ ID NO: 38 | 29% |
| Sorghum bicolor | Sb01g023290 | SEQ ID NO: 39 | SEQ ID NO: 40 | 30% |
| Zea mays | LOC100191257 | SEQ ID NO: 41 | SEQ ID NO: 42 | 31% |

In Table 2, columns for “Identities” indicate percentages accounted for by identical residues including analogous residues found by comparison of amino acids, which were calculated by BlastP search.

The above-explained genes belonging to the TIFY family, such as the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene, and homologous genes thereof are introduced into plants, or the expression control regions of the genes endogenous to the plants are modified to enhance the expression of the genes, so that the disease resistance of the plants can be significantly improved. Disease resistance in plants is not particularly limited, and can be evaluated by conventionally known methods. For example, specific leaves of a plant to be evaluated are sprayed with a solution containing pathogenic bacteria. After a predetermined time period, the number of lesions is measured and then disease resistance can be evaluated based on the number of lesions. According to the present invention, the number of lesions decreases by 10% or more, preferably 20% or more, more preferably 30% or more, further preferably 40% or more, more preferably 50% or more, and most preferably 60% or more, for example, compared with wild-type plants.

Also, an example of a technique for introducing the above genes into plants is a technique for introducing an expression vector in which exogenous genes are placed under control of a promoter that enables expression within plant bodies. An example of a technique for modifying the expression control regions of the endogenous genes is a technique for modifying promoters of the above genes endogenous to target plants.

Among the above gene group, disease resistance in a plant is preferably improved by introducing one gene selected from the group consisting of, in particular, the OsTIFY11d gene, the OsTIFY11a gene, the OsTIFY11e gene, and the OsTIFY10a gene, and homologous genes thereof into the plant, so as to enhance the expression of the gene. Such a plant, in which the expression of one gene selected from the group consisting of the OsTIFY11d gene, the OsTIFY11a gene, the OsTIFY11e gene, and the OsTIFY10a gene, and homologous genes thereof is enhanced, can be grown and can exhibit disease resistance equivalent to wild-type plants.

<Expression Vector>

An expression vector is constructed to contain a promoter that enables gene expression in a plant body and the above gene. As vector bases for expression vectors, conventionally known various vectors can be used. For example, a plasmid, a phage, a cosmid, or the like can be used, which can be appropriately selected depending on plant cells into which it is introduced or introduction methods. Specific examples of vectors include pBR322, pBR325, pUC19, pUC119, pBluescript, pBluescriptSK, and pBI vectors. In particular, when a method for introducing a vector into a plant body is a method using Agrobacterium, a pBI binary vector is preferably used. Specific examples of the pBIbinary vector include pBIG, pBIN19, pBI101, pBI121, and pBI221.

Promoters are not particularly limited, as long as they enable the expression of the above gene within plant bodies. Known promoters can be appropriately used. In particular, as a promoter, a promoter capable of constantly inducing the expression of a gene placed at a site downstream within a plant body can be preferably used. Examples of such a promoter include a cauliflower mosaic virus 35S promoter (CaMV35S), various actin gene promoters, various ubiquitin gene promoters, a nopaline synthase gene promoter, a tobacco PR1a gene promoter, a tomato ribulose 1,5-diphosphate carboxylase.oxidase small subunit gene promoter, and a unpin gene promoter. Of these, cauliflower mosaic virus 35S promoter, actin gene promoter, or ubiquitin gene promoter can be used more preferably. The use of each of the above promoters enables strong expression of an arbitrary gene when introduced into plant cells.

In addition, as a promoter, a promoter having a function to cause site-specific gene expression in plants can also be used. As such a promoter, any conventionally known promoter can be used. The above gene(s) is expressed in a site-specific manner using such a promoter, and thus disease resistance in plant organs and/or tissues in which the gene is expressed can be improved compared with wild-type plants.

In addition, an expression vector may contain, in addition to a promoter and the above gene, other DNA segments. Examples of such other DNA segments include, but are not particularly limited to, a terminator, a selection marker, an enhancer, and a nucleotide sequence for enhancing translation efficiency. The above recombinant expression vector may further have a T-DNA region. The T-DNA region can enhance gene transfer efficiency particularly when the above recombinant expression vector is introduced into plant bodies using Agrobacterium.

A transcription terminator is not particularly limited as long as it has functions of a transcription termination site, and may be a known transcription terminator. For example, specifically, the transcription termination region (Nos terminator) of a nopaline synthase gene, the transcription termination region (CaMV35S terminator) of cauliflower mosaic virus 35S, and the like can be preferably used. Of these, the Nos terminator can be more preferably used, in the above recombinant vector, a transcription terminator is placed at an appropriate position. Thus, after introduction into plant cells, a phenomenon such that an unnecessarily long transcript is synthesized and a strong promoter decreases the number of plasmid copies can be prevented from occurring.

As a transformant selection marker, a drug resistance gene can be used, for example. Specific examples of such a drug resistance gene include hygromycin, bleomycin, kanamycin, gentamicin, and chloramphenicol resistance genes. Plant bodies that grow in medium containing the above antibiotics are selected using these markers, so that transgenic plant bodies can be easily selected.

An example of a nucleotide sequence for enhancing translation efficiency is a tobacco mosaic virus-derived omega sequence. The omega sequence is placed in the untranslated region (5′UTR) of a promoter, so that translation efficiency for the above fusion gene can be increased. As described above, the above recombinant expression vector can contain various DNA segments depending on the purposes thereof.

A method for constructing a recombinant expression vector is also not particularly limited. The above promoter, the above gene(s), and if necessary the above other DNA segments are introduced into an appropriately selected vector base in a predetermined order. For example, the above gene is ligated to a promoter (if necessary, a transcription terminator and the like) to construct an expression cassette, and then the expression cassette is introduced into a vector. Upon construction of such an expression cassette, for example, cleavage sites of each DNA segment are prepared to have protruding ends complementary to each other. Through reaction with a ligation enzyme, the order of DNA segments can be specified, in addition, when an expression cassette contains a terminator, a promoter, the above gene, and a terminator should be aligned in this order from upstream. Furthermore, reagents for constructing an expression vector; that is, types of restriction enzyme, ligation enzyme, or the like are also not particularly limited. Commercially available reagents may be appropriately selected and used.

<Transformation>

The above-described expression vector is introduced into a target plant by a general transformation method. Methods for introducing (transformation method) an expression vector into plant cells are not particularly limited. Conventionally known appropriate transformation methods can be used depending on plant cells. Specifically, for example, a method using Agrobacterium and a method for directly introducing an expression vector into plant cells can be employed. As such a method using Agrobacteriurn, for example, a method described in Bechtold, E., Ellis, J. and Pelletier, G. (1993) in Planta Agrobacterium-mediated gene transfer by infiltration of adult Arabidopsis plants. C.R. Acad. Sci, Paris Sci. Vie, 316, 1194-11.99., or, Zyprian E, Kado Cl, Agrobacterium-mediated plant transformation by novel mini-T vectors in conjunction with a high-copy vir region helper plasmid. Plant Molecular Biology, 1990, 15(2), 245-256. can be employed.

Examples of a method for directly introducing an expression vector into plant cells, which can be employed herein, include the microinjection method, the electroporation method, the polyethylene glycol method, the particle gun method, the protoplast fusion method, and the calcium phosphate method.

In addition, when such a method for directly introducing DNA into plant cells is employed, DNA to be sufficiently used herein contains transcriptional units required for the expression of a target gene, such as a promoter and a transcription terminator, and the target gene. Vector functions are not essential. Moreover, even DNA containing only the protein coding region of a target gene, but having no transcriptional unit may be used herein, as long as it can be integrated into the transcriptional unit of a host and enables the expression of the target gene.

Examples of plant cells, into which the above expression vector or expression cassette containing no expression vector but containing a target gene is introduced, include cells of each tissue in plant organs (e.g., flower, leaf, and root), callus, and suspension-cultured cells, and the like. Here, an appropriate expression vector can be adequately constructed according to the type of plant bodies to be produced. A general expression vector is constructed in advance and then may be introduced into plant cells.

Target plants into which an expression vector is introduced, in other words, target plants to which disease resistance is conferred are not particularly limited. Specifically, the above gene is expressed, so that the effect of conferring disease resistance to all plant bodies can be expected. Examples of target plants include, but are not limited to, dicotyledons and monocotyledons, such as plants belonging to the family Brassicaceae, the family Gramineae, the family Solanaceae, the family Leguminosae, the family Slalicaceae, or the like (see below).

The family Brassicaceae: Arabidopsis thaliana (Arabidopsis thaliana), rapeseed (Aburana) (Brassica rapa, Brassica napus), cabbage (Brassica oleracea var. capitata), rapeseed (Natane) (Brassica rapa, Brassica napus), qing-geng-eai (Brassica rapa var. chinensis), turnip (Brassica rapa var. rapa), nozawana (Brassica rapa var. hakabura), potherb mustard (Brassica rapa var. lancinifolia), komatsuna (Brassica rapa var, peruviridis), Chinese cabbage (Brassica rapa var. chinensis), radish (Brassica Raphanus sativus), wasabi (Wasabia japonica), etc.

The family Solanaceae: tobacco (Nicotiana tabacum), eggplant (Solanum melongena), potato (Solarteurn tuberosuin), tomato (Lycopersicon lycopersicum), pepper (Capsicum annuum), petunia (Petunia), etc.

The family Leguminosae: soybean (Glycine max), pea (Pisum sativum), fava bean (Vicia faba), Japanese wisteria (Wisteria floribunda), peanut (Arachis hypogaea), Lotus japonicus (Lotus corniculatus var. japonicus), common bean (Phaseolus vulgaris), azuki (Vigna angularis), acacia (Acacia), etc.

The family Compositae: Chrysanthemum (Chrysanthemum morifolium), sunflower (Helianthus annuus), etc.

The family Arecaceae: oil palm (Elaeis guineensis, Eiaeis oleifera), coconut (Cocos nucifera), date palm (Phoenix dactylifera), wax palm (Copernicia), etc.

The family Anacardiaceae: hazenoki (Rhus succedanea), cashew (Anacardium occidentale), poison oak (Toxicodendron vernicifluum), mango (Mangifera indica), pistachio (Pistacia vera), etc.

The family Cucurbitaccae: pumpkin (Cucurbita maxima, Cucurbila moschata, Cucurbita pepo), cucumber (Cucumis sativus), trichosanthes (karasu ant) (Trichosanthes cucumeroides), gourd (Lagenania siceraria var. gourda), etc.

The family Rosaceae: almond (Arnygdalus communis), rose (Rosa), strawberry (Fragaria), cherry (Prunus), apple (Malus pumila var. domestica), ete.

The family Caryophyllaceae: carnation (Dianthus caryophyllus) etc.

The family Salicaceae: poplar (Populus trichocarpa, Populus nigra, Popular tremula), etc.

The family Myrtaceae: eucalyptus (Eucalyptus camaldulensis, Eucalyptus grandis), etc.

The family Gramincae: corn (Zea mays), rice (Oryza, sativa), barley (Hordeum vulgare), wheat (Trilieum aestivuin), bamboo (Phyilostachys), sugarcane (Saccharum offieinarum), napier grass (Pennisetum pupureum), erianthus (Erianthus ravenae), Japanese silver grass (Miscanthus virgatum), sorghum (Sorghum), switchgrass (Panicum), etc.

The family Liliaceae: tulip (Tulipa), lily (Lilium), etc.

Of these, plants belonging to the family Grarnineae such as rice, wheat, barley, sugarcane, and corn are preferable subjects of the present invention. In particular, when any one of the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene is used, rice is preferably a subject since the origin of these genes is rice. In addition, when the above-described homologous gene is used, a plant of the same species as the species from which the homologous gene (to be used herein) is derived is preferably a subject.

However, plants, to which any one of genes belonging to the TIFY family, such as the OsTIFY11a gene, the OsTIFY11b gene, the OsTIFY11c gene, the OsTIFY11d gene, the OsTIFY11e gene, the OsTIFY11f gene, the OsTIFY11g gene, the OsTIFY10a gene, the OsTIFY10b gene, and the OsTIFY10c gene, and homologous genes thereof is introduced, are not limited to plants of the same species and may be plants of different species. Specifically, rice-derived genes belonging to the TIFY family may be introduced into and expressed in a dicotyledon, for example.

As described above, the disease resistance of a plant is significantly improved by introducing a gene belonging to the TIFY family. Therefore, the present invention can prevent a yield amount from decreasing due to disease induced by pathogenic bacteria or the like, and can improve plant productivity. For example, the above gene is introduced into a plant that can be used as a raw material for a biofuel, so that the productivity of the plant can be improved. As a result, biofuel production cost can be lowered.

<Other Steps and Other Methods>

After the above transformation, transformants may be selected based on drug resistance (e.g., drug resistance such as hygromycin resistance) using a drug resistance marker that has been introduced together with the above gene. Alternatively, disease resistance of a plant body itself or the same of an arbitrary organ or tissue is determined, and then those having significantly improved disease resistance compared with wild-type plants may be selected as transformants.

In addition, progeny plants can be obtained from transgenic plants obtained by transformation, according to a standard method. Specifically, progeny plants retaining the phenotype of disease resistance that is acquired by enhancing the expression of the above gene can be obtained according to a standard method.

In addition, plant bodies in the present invention include at least any one of plant individuals that have grown, plant cells, plant tissues, calluses, and seeds. Specifically, in the present invention, those in a state that can be finally grown to plant individuals are all regarded as plant bodies. The above plant cells further include plant cells in various forms. Examples of such plant cells include suspension-cultured cells, protoplasts, and leaf sections. These plant cells are caused to proliferate and differentiate, so that plant bodies can be obtained. Moreover, regeneration of plant bodies from plant cells can be carried out using a conventionally known method depending on plant cell types.

<Disease Resistance>

As explained above, according to the present invention, the above specific gene belonging to the TIFY family is introduced into a plant, or the expression control region of the gene endogenous to the plant is modified to enhance the expression of the gene, and thus disease resistance is improved compared with wild-type plant bodies.

The term “disease resistance” in the present invention refers to resistance to disease that is developed as a result of infection with pathogenic microorganisms or viruses. Examples of pathogenic microorganisms include, but are not particularly limited to, filamentous fungi, viruses, and bacteria infecting plants. Examples of filamentous fungi infecting plants include fungi belonging to the genus Magnaporthe such as Magnaporihe grisea (namely, a blast pathogen), fungi of the genus Botrytis such as Botrytis cinerea, fungi of the genus Aspergillus such as Aspergillus flavus, fungi of the genus Colletotrichum such as Colletotrichum acutatum, fungi of the genus Fusarium such as Fusarium ovysporurn, fungi of the genus Alternaria such as Alternaria aliernata, fungi of the genus Rhizoctonia such as Rhizoctonia solani, fungi of the genus Sclerotium such as Sclerotium rolfsii.

Furthermore, examples of pathogenic microorganisms include microorganisms of the genus Taphrina, microorganisms of the genus Blumeria, microorganisms of the genus Cystotheca, microorganisms of the genus Erysiphe, microorganisms of the genus Golovinomyces, microorganisms of the genus Phyllactinta, microorganisms of the genus Podosphaera, microorganisms of the genus Sawarfaea, microorganisms of the genus Ceratocystis, microorganisms of the genus Monosporascus, microorganisms of the genus Claviceps, microorganisms of the genus Calonectria, microorganisms of the genus Gibbereila, microorganisms of the genus Haematonecria, microorganisms of the genus Nectria, microorganisms of the genus Neonectria, microorganisms of the genus Glomerella, microorganisms of the genus Cryphonectria, microorganisms of the genus Diaporthe, microorganisms of the genus Valsa, microorganisms of the genus Pestalosphaeria, microorganisms of the genus Roseilinia, microorganisms of the genus Ciborinia, microorganisms of the genus Ovulinia, microorganisms of the genus Aionilinia, microorganisms of the genus Diplocarpon, microorganisms of the genus Elsinoe, microorganisms of the genus Cochliobolus, microorganisms of the genus Didymella, microorganisms of the genus Pleaspora, microorganisms of the genus Venturia, microorganisms of the genus Mycosphaerelia, microorganisms of the genus Helicobasidium, microorganisms of the genus Ustilago, microorganisms of the genus Tilletia, microorganisms of the genus Exobasidium, microorganisms of the genus Coleosporium, microorganisms of the genus Cronartium, microorganisms of the genus Melampsora, microorganisms of the genus Phakopsora, microorganisms of the genus Phragmidium, microorganisms of the genus Gymnosporangium, microorganisms of the genus Uromyces, microorganisms of the genus Blastospora, microorganisms of the genus Thanatephorus, microorganisms of the genus Armillaria, microorganisms of the genus Erythricium, microorganisms of the genus Perenniporia, microorganisms of the genus Ganoderma, microorganisms of the genus Phoma, microorganisms of the genus Pyrenochueta, microorganisms of the genus Phomopsis, microorganisms of the genus Gloeodes, microorganisms of the genus Tubakia, microorganisms of the genus Ascochyta, microorganisms of the genus Lasiediplodia, microorganisms of the genus Pestalotiopsis, microorganisms of the genus Ateroconium, microorganisms of the genus Oidiopsis, microorganisms of the genus Verticillium, microorganisms of the genus Penicillium, microorganisms of the genus Cladosporium, microorganisms of the genus Corynespora, microorganisms of the genus Fulvia, microorganisms of the genus Cereospera, microorganisms of the genus Pseudocercospora, microorganisms of the genus Aphanomyces, microorganisms of the genus Phytophthora, microorganisms of the genus Pythium, microorganisms of the genus Albugo, microorganisms of the genus Peronospora, microorganisms of the genus Piasmopora, microorganisms of the genus Rhizopus, and microorganisms of the genus Choanephora.

Specifically, in the present invention, when the expression of the above gene is enhanced in rice plants, the rice plants can acquire resistance to at least one type of pathogenic microorganism selected from the group consisting of rice blast pathogen (scientific name: Magnaporthe grisea), rice false smut pathogen Claviveps virens (Ustilaginoidea virens), rice bakanae bye (disease caused by (Gibbereila fujikuroi infecting rice seedlings) pathogen Gibberella fujikuroi, rice brown spot pathogen Cochliobolus miyabeanus (Bipolaris oryzae), rice sheath blight pathogen Thanatephorus cucumeris (Rhizoctonia solani), rice bacterial leaf spot pathogen Xanthomonas oryzae pv. oryzae, rice bacterial brown stripe pathogen Acidovorax avenae subsp. avenae, rice bacterial grain rot pathogen Burkholderia glumae, and rice red stripe disease pathogen Microbacterium sp., for example.

Furthermore, in the present invention, when the expression of the above gene is enhanced in corn plants, the corn plants can acquire resistance to at least one type of pathogenic microorganism selected from the group consisting of corn brown spot (goma hagare hyo) pathogen Cochliobolus heterostrophus (Bipolaris maydis), corn zonate leaf spot pathogen Gloeocercospora sorghi, corn brown spot (kappan hyo) Kahatiella zeae, corn brown stripe downy mildew pathogen Sclerophthora rayssiae, corn Pythium stalk rot pathogen Pythium aphanidermatum, corn smut pathogen Ustilago maydis, corn sheath blight pathogen Rhizoctonia solani, corn seed rot and damping-off pathogen Pythium spp., corn tropical rust pathogen Puccinia polysora, and corn root rot pathogen (Pythium graminicola Subramanian).

### EXAMPLES

Hereinafter, the present invention is described in greater detail with reference to the examples below, although the technical scope of the present invention is not limited to these examples.

### Example 1

### Construct Preparation Method

In this example, to produce a line highly expressing OsTIFY11d, PCR was carried out using single-stranded cDNA prepared from Nipponbare leaf tissue as a template so as to amplify the cDNA of OsTIFY11d. PCR was carried out using a reaction solution with the following composition.

TABLE 3

| | | |
|---|---|---|
| 2 × KOD buffer | 12.5 | μl |
| 2 mM dNTP Mix | 5 | μl |
| 10 μM forward primer | 0.8 | μl |
| 10 μM reverse primer | 0.8 | μl |
| Single-stranded cDNA | 2 | μl |
| KOD (TOYOBO) | 0.4 | μl |
| DMSO | 2.5 | μl |
| total | 25 | μl |

In this PCR, the following primer set for amplification of OsTIFY11d was used.

Fw:
(SEQ ID NO: 43)
5′-GGTACCCACACGCAAGCTTCGCAGCG-3′
Rv:
(SEQ ID NO: 44)
5′-GAATTCGACAACAGTCTTTCCAGTCTTTTGAG-3′

PCR was carried out with the following program. Specifically, PCR was carried out by, after thermal denaturation at 95° C. for 3 minutes, repeating 35 times a cycle of 30 seconds of thermal denaturation at 95° C., 30 seconds of annealing at 57° C., and 100 seconds of elongation reaction at 68° C. PCR products were separated with 1.5% agarose gel, stained with ethidium bromide, excited by UV, and then subjected to detection procedures. The obtained PCR product was cloned to a pCR2.1-TOPO vector (Invitrogen). For production of a line highly expressing OsTIFY11d, an OsTIFY11d expression vector was constructed by inserting a fragment obtained by digestion of OsTIFY1 id cDNA with Kpn I/EcoR I into a pUBIN-ZH1 binary vector that had been similarly digested, and then the vector was used.

For preparation of a line with suppressed expression of OsTIFY11d, PCR was carried out using single-stranded cDNA prepared from Nipponbare leaf tissue as a template, so as to amplify a partial sequence (406 bp) of the OsTIFY11d gene, PCR was carried out using a reaction solution with the following composition.

TABLE 4

| | |
|---|---|
| 10 × Ex taq buffer | 2.5 μl |
| 2.5 mM dNTP Mix | 2.5 μl |
| 10 μM forward primer | 0.5 μl |
| 10 μM reverse primer | 0.5 μl |
| Single-stranded cDNA | 2 μl |
| Ex taq (Takara) | 0.4 μl |
| total | 25 μl |

In this PCR, the following primer set for amplification of an OsTIFY11d fragment was used.

Fw:
(SEQ ID NO: 45)
5′-AACGAGCGAACCATACAAGAAG-3′
Rv:
(SEQ ID NO: 46)
5′-ACAACAGTCTTTCCAGTCTTTTGAG-3′

PCR was carried out with the following program. Specifically, PCR was carried out by, after thermal denaturation at 95° C. for 3 minutes, repeating 35 times a cycle of 30 seconds of thermal denaturation at 95° C., 30 seconds of annealing at 62° C., and 60 seconds of elongation reaction at 72° C. PCR products were separated with 1.5% agarose gel, stained with ethidium bromide, excited by UV, and then subjected to detection procedures. The obtained PCR product was cloned to a pCR8/GW/TOPO vector (Invitrogen). For production of a line with suppressed expression of OsTIFY11d, a vector with suppressed expression of OsTIFY11d was constructed by inserting an OsTIFY11d fragment (cloned into pCR8/GW/TOPO) to a pANDA vector by LR reaction, and then used.

<Transformation Method>

Fully ripened rice (cultivar: Nipponbare) seeds were dehusked, and then sterilized with a 70% ethanol and sodium hypochlorite aqueous solution (available chlorine 4% to 6%). After being washed with sterile water, seeds were placed in N6D medium, and then cultured at 30° C. under continuous light for 3 weeks, so as to induce calluses. The induced calluses derived from scutellum were transferred to fresh N6D medium, and then cultured for 5 days.

The Agrobacterium cells into which a plant expression vector plasmid had been introduced were applied to AB medium and then cultured at 28° C. in the dark. Agrobacterium cells that had grown were collected and then suspended in AAM medium supplemented with acetosyringone. The cultured calluses were soaked in the Agrobacterium suspension, an excess Agrobacterium suspension was removed, and then the calluses were placed in 2N6AS medium. Agrobacterium cells were co-cultured together with calluses at 25° C. in the dark for 2.5 days for infection. The infected calluses were washed with sterile water to remove Agrobacterium cells. After removal of excess water from the calluses, the calluses were placed in N6D medium supplemented with hygromycin and carbenicillin, and then cultured at 30° C. under continuous light for 3 weeks. Calluses that had proliferated were transferred to MSNK medium and cultured at 28° C. under continuous light. Individuals that had re-differentiated were transferred to rooting medium to accelerate root growth. After plant bodies had grown, they were transferred to pots containing culture soil for growing seedlings and then cultivated in a closed system greenhouse. The composition of the medium used herein is as follows.

TABLE 5
Callus induction medium (N6D)

| | | |
|---|---|---|
| CHU N6 salts (Sigma) | 4 | g |
| N6 vitamin (×100) | 10 | ml |
| myo-inositol | 100 | mg |
| casamino acid | 300 | mg |
| proline | 2.8 | g |
| 2,4-D (0.2 mg/ml) | 10 | ml |
| sucrose | 30 | g |

(After dissolution in about 800 ml of distilled water, the pH was adjusted with NaOH aqueous solution to pH 5.8, and then 3 g of gellan gum was added to adjust the volume to 1000 ml. After mixing, autoclave sterilization was carried out at 121° C. for 20 minutes.)

TABLE 6
N6 vitamin (×100)

| | | |
|---|---|---|
| glycine | 20 | mg |
| nicotinic acid | 5 | mg |
| pylidoxine, | 5 | mg |
| hydrochloride | | |
| thiamine | 20 | mg |
| H₂O | to 100 | ml |

TABLE 7
AB medium

| | | |
|---|---|---|
| K₂HPO₄ | 3 | g |
| NaH₂PO₄•2H₂O | 1 | g |
| NH₄Cl | 1 | g |
| KCl | 150 | mg |
| CaCl₂•2H₂O | 12 | mg |
| FeSO₄•7H₂O | 2.5 | mg |
| Glucose | 5 | g |

(After dissolution in about 800 ml of distilled water, the pH was adjusted with NaOH aqueous solution to pH 7.2, and then the solution was diluted to 1000 ml. Agar (15 g) was added. After 20 minutes of autoclaving at 121′C, 1.22 ml of 1 M MgSO₄.7H₂O sterilized with a filter was added.)

TABLE 8
AAM medium (liquid medium for suspension of
Agrobacterium tumefaciens)

| | | |
|---|---|---|
| 1000 × AA1 | 1 | ml |
| 1000 × AA2 | 1 | ml |
| 1000 × AA3 | 1 | ml |
| 1000 × AA4 | 1 | ml |
| 1000 × AA5 | 1 | ml |
| 200 × AA6 | 5 | ml |
| 100 × AA sol. | 10 | ml |
| casamino acid | 0.5 | g |
| sucrose | 68.5 | g |
| glucose | 36 | g |
| L-glutamine | 900 | mg |
| L-aspartate | 300 | mg |
| KCl | 3.0 | g |

(After dissolution in about 800 ml of sterile water, the pH was adjusted with NaOH aqueous solution to pH 5.2. The solution was diluted to 1000 ml and then autoclaving was carried out at 121° C. for minutes. One (1) ml of 10 mg/ml acetosyringone (3′,5′-Dimethoxy-4′-hydroxyacetophenone, in DMSO) was mixed immediately before use.)

TABLE 9
AA stock solution (10 mL)

| | | | |
|---|---|---|---|
| 1000 × AA1 | MnSO₄•H₂O | 100 | mg |
| | H₃BO₄ | 30 | mg |
| | ZnSO₄•7H₂O | 20 | mg |
| | CuSO₄•5H₂O | 0.25 | mg |
| | CoCl₂•6H₂O | 0.25 | mg |
| | KI | 7.5 | mg |
| | H₂O | to 10 | ml |
| 1000 × AA2 | CaCl₂•2H₂O | 1.5 | g |
| | H₂O | to 10 | ml |
| 1000 × AA3 | MgSO₄•7H₂O | 2.5 | g |
| | H₂O | to 10 | ml |
| 1000 × AA4 | Fe-EDTA | 0.4 | g |
| | H₂O | to 10 | ml |
| 1000 × AA5 | NaH₂PO₄•2H₂O | 1.5 | g |
| | H₂O | to 10 | ml |
| 1000 × AA6 | myo-inositol | 10 | g |
| | nicotinic acid | 100 | mg |
| | pyridoxine, | 100 | mg |
| | hydrochloride | | |
| | thiamine | 1000 | mg |
| | H₂O | to 500 | ml |
| 100 × AAsol. | L-arginine | 1.6 | g |
| | glycine | 75 | mg |
| | H₂O | to 100 | ml |

TABLE 10
Co-culture medium (2N6AS)

| | | |
|---|---|---|
| CHU N6 salts (Sigma) | 4 | g |
| N6 vitamin (×100) | 10 | ml |
| myo-inositol | 100 | mg |
| casamino acid | 300 | mg |
| 2,4-D (0.2 mg/ml) | 10 | ml |
| sucrose | 30 | g |
| glucose | 10 | g |

(After dissolution in about 800 ml of sterile, water, the pH was adjusted with NaOH aqueous solution to pH 5.2, 4 g of gellan gum was added, the solution was diluted to 1000 ml, and then autoclaving was carried out at 121° C. for 20 minutes. After cooling, 1 ml of 10 mg/ml acetosyringone dissolved in DMSO was mixed.)

TABLE 11
Redifferentiation medium (MSNK)

| | | |
|---|---|---|
| MS salts (Nippon Seiyaku) | 4.6 | g (1 pack) |
| MS vitamin (×100) | 10 | ml |
| myo-inositol | 100 | mg |
| casamino acid | 2 | g |
| kinetine(0.1 mg/ml) | 20 | ml |
| NAA (0.2 mg/ml) | 1 | ml |
| sucrose | 30 | g |
| sorbitol | 30 | g |

(After dissolution in about 800 ml of sterile water, the pH was adjusted with NaOH aqueous solution to pH 5.8, 3 g of gellan gum was added, and then the solution was diluted to 1000 ml. After mixing, autoclaving was carried out at 121° C. for 20 minutes. After cooling, 1 ml of carbenicillin (250 mg/ml) and 1 ml of hygromycin (50 mg/ml) were added.

TABLE 12
MS vitamin (×1000029

| | | |
|---|---|---|
| Glycine | 20 | mg |
| nicotinic acid | 50 | mg |
| pyridoxine, | 100 | mg |
| hydrochloride | | |
| thiamine | 100 | mg |
| H₂O | to 100 | ml |

<Expression Analysis>

Leaf tissues of transformants and a wild-type line grown in granular culture soil were frozen and crushed, and then total RNA was extracted using a Sepasol reagent (nakarai). The extracted total RNA (10 μg) was treated with DNase I (Takara Bio) to degrade DNA mixed therewith, and then purified by phenol/chloroform/isoamyl alcohol (25:24:1) extraction and ethanol precipitation. Single-stranded cDNA was synthesized from the total RNA. Reverse transcription reaction was carried out using a PrimeScript RT reagent Kit (Takara Bio). The obtained single-stranded cDNA was diluted 10 fold and then quantitative real time PCR was carried out using the resultant as a template, PCR was carried out using SYBR Premix Ex Taq (Takara Bio) and a reaction solution with the following composition.

TABLE 13

| | |
|---|---|
| 2 × SYBR Premix ExTaq | 12 μl |
| 5 μM forward primer | 2 μl |
| 5 μM reverse primer | 2 μl |
| Single-stranded cDNA | 2 μl |
| total | 25 μl |

PCR was carried out with the following program. Specifically, PCR was carried out by, after 30 seconds of thermal denaturation at 95° C., repeating 40 times a cycle of 5 seconds of thermal denaturation at 95° C. and 45 seconds of elongation reaction at 60° C. The following primer set for analyzing OsTIFY11d expression was used.

Fw:
(SEQ ID NO: 47)
5′-TGAAAGATGAGCCGGCGAC-3′
Rv:
(SEQ ID NO: 48)
5′-TCCCTTTGTGATATTCTCCTCCTCT-3′

An ABI Prism 7500 Sequence Detection system (PE Applied Biosystems) was used for reaction and detection. A calibration curve was produced using serially diluted cDNA samples. Ubiquitin (Os03g0234200) was used as an internal standard gene and then correction was made on the basis of the expression level. The following primer set for amplification of Ubiquitin was used.

Fw:
(SEQ ID NO: 49)
5′-AACCAGCTGAGGCCCAAGA-3′
Rv:
(SEQ ID NO: 50)
5′-ACGATTGATTTAACCAGTCCATGA-3′

<Blast Pathogen Inoculation Method>

[Spray Inoculation]

Water was supplied to the fully ripen seeds of the wild-type line and the transgenic line for 2 days. The obtained germinating seeds were seeded in a cell sheet (2.5 cm×2.5 cm×4 cm) containing granular culture soil. Seedlings grown for about 2 weeks within a closed system greenhouse (25° C./20° C., natural light) were transferred into plastic tubes containing a fertilizer solution (Chiyoda, 0.2%). Seedlings were further grown and then plant bodies that had developed the 5^{th} leaves were used for an inoculation test.

The blast pathogen used herein was Magnaporthe grisea race 007, Plant bodies were left to stand within an inoculation box, and then spray inoculated with a blast pathogen spore solution (2.5×10⁵ spores/ml). Rice plants were left to stand within the inoculation box in the dark under conditions of 100% moisture for 20 hours. Rice plants were then removed from the inoculation box and then cultivation thereof was continued within the closed system greenhouse. Five days after inoculation, the numbers of susceptible lesions that had appeared on the 5^{th} leaves were measured and then the degrees of onset were evaluated.

[Drop Inoculation]

Water was supplied to the fully ripen seeds (T1 seeds in the case of the transgenic line) of the wild-type line and the transgenic line for 2 days. The obtained germinating seeds were seeded in plastic cups (250 ml) containing granular culture soil and then grown within a closed-system greenhouse (25° C./20° C. natural light). Paper towel soaked in water was placed within a transparent container with a lid, laminae of the 4^{th} or the 5^{th} leaves at almost the same growth stage were removed from plant bodies grown within the closed-system greenhouse and then aligned over the paper towel. A blast pathogen used herein was Magnaporthe grisea race 007. A spore suspension was prepared to have a concentration of about 2.5×10⁵ spores/ml. The blast pathogen spore suspension was added dropwise each) (3 μl each) onto aligned laminae at appropriate intervals. After inoculation, the container was covered with the lid for infection in such a place avoiding direct sunlight. During infection, water was added adequately so as to avoid drying. Five (5) days after inoculation, the lengths of lesions were measured and thus the degrees of onset were evaluated.

<Measurement of Growth>

Transgenic T1 seeds were dehusked and then treated with a 70% ethanol and sodium hypochlorite aqueous solution (available chlorine 4% to 6%) and then washed with sterile water for surface sterilization. The seeds were seeded in Murashige-Skoog medium (containing 2% sucrose and 0.3% Gellan Gum) containing 50 μg/ml hygromycin, and then grown at 25′C under continuous light for 14 days. Plant bodies that had exhibited resistance to hygromycin and grown were transferred to 250-ml cups containing granular culture soil and then grown within a closed-system greenhouse (25° C./20° C., natural light) until the ripening period. Ripen plant bodies were measured for plant height, stem length, and flag leaf length and then the lengths were compared.

<Results>

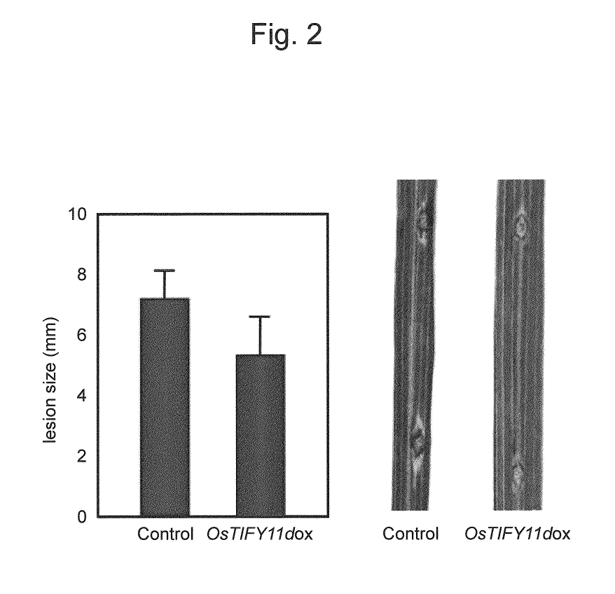

FIG. 1 shows the results of spray inoculation with the blast pathogen and then measuring the numbers of susceptible lesions that had appeared on the 5^{th} leaves at 5 days after inoculation, and photographs of the 5^{th} leaves. FIG. 2 shows the results of drop inoculation with the blast pathogen and measuring the progress of lesions at 5 days after inoculation, and photographs of the leaves.

As shown in FIG. 1 and FIG. 2, in the case of the line highly expressing OsTIFY11d (produced in the Example), the number of lesions resulting from infection with the blast pathogen was significantly lower than that of wild-type plants (by about 61%), and the progress of lesions infected with the blast pathogen was significantly lower than that of wild-type plants. It was revealed by the results that the line highly expressing OsTIFY11d (produced in the Example) had good resistance to blast.

Furthermore, FIG. 3 shows the results of measuring the growth of the line highly expressing OsTIFY11d (produced in the example). In addition, FIG. 3 shows in the upper left the results of measuring the full lengths of plant bodies (11 days after seeding), and in the upper middle and the right the photographs are of plant bodies (11 days after seeding and ripening period). In addition, FIG. 3 shows in the lower left the results of measuring the full lengths of plant bodies in the ripening period, in the lower middle the results of measuring the stem lengths of plant bodies in the ripening period, and in the lower right the results of measuring the lengths of flag leaves of plant bodies in the ripening period.

As shown in FIG. 3, the line highly expressing OsTIFY11d (produced in the example) was found to exhibit accelerated growth compared with wild-type plants.

Specifically, it was revealed by the example that high-level expression of a specific gene belonging to the TIFY family can confer disease resistance to the plant or improve disease resistance of the plant, and can accelerate plant growth.

### Example 2

In this example, according to the method for producing the line highly expressing OsTIFY11d, as explained in Example 1, a line highly expressing OsTIFY10a, a line highly expressing OsTIFY11a, and a line highly expressing OsTIFY11e were produced. Unlike Example 1, the following primer sets were used as a primer set for amplification of OsTIFY10a, a primer set for amplification of OsTIFY11a, and a primer set for amplification of OsTIFY11e.

Primer set for amplification of OsTIFY10a:

Fw:
(SEQ ID NO: 57)
5′-TTTGGTACCACTCAGAGACAGACAAGGACGAG-3′
Rv:
(SEQ ID NO: 58)
5′-TTGACTAGTATGAGGTTTCTTGGGTTGTACTG-3′

Primer set for amplification of OsTIFY11a:

Fw:
(SEQ ID NO: 59)
5′-TGGTACCTATGTGATCAGCGACGTACAGTACAG-3′
Rv:
(SEQ ID NO: 60)
5′-TGAATTCCAATTCAGTCCCCATAGGAATCG-3′

Primer set for amplification of OsTIFY11e:

Fw:
(SEQ ID NO: 61)
5′-GAAGGTACCCCGAGATTTTCTCCGAACAC-3′
Rv:
(SEQ ID NO: 62)
5′-CTAGAGCTCCTTGTGACAGATAGGAATAATCGTG-3′

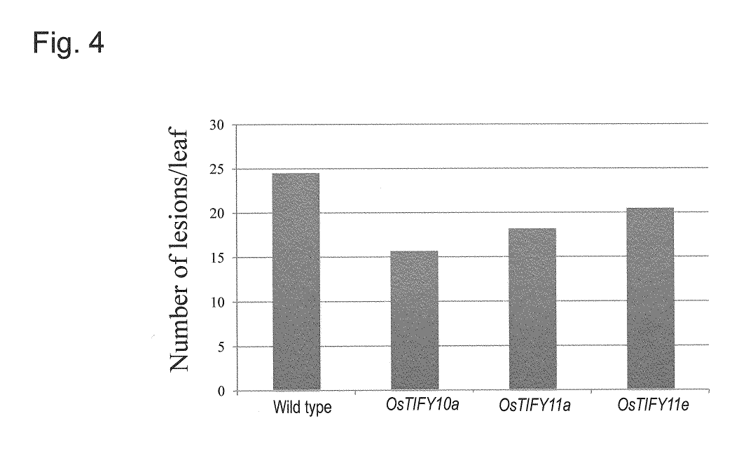

In a manner similar to Example 1, transformants grown in granular culture soil were spray inoculated with a blast pathogen and then the numbers of susceptible lesions that appeared on the 5^{th} leaves at 5 days after inoculation were measured. FIG. 4 shows the results.

As shown in FIG. 4, in the case of the line highly expressing OsTIFY10a, the line highly expressing OsTIFY11a, and the line highly expressing OsTIFY11e, the numbers of lesions due to infection with the blast pathogen decreased by about 36%, about 26%, and about 16%, respectively, compared with wild-type plants. It was revealed by the results that the line highly expressing OsTIFY10a, the line highly expressing OsTIFY11a, and the line highly expressing OsTIFY11e (produced in the example) had good resistance to blast.

It was revealed by the results of Example 1 and Example 2 that high-level expression of a gene belonging to the rice THY family can confer resistance to diseases resulting from the blast pathogen or the like, to plants.

All publications, patents, and patent applications cited herein are incorporated herein by reference in their entirety.

1. A method for conferring disease resistance to a plant, comprising a step of introducing at least one of the genes belonging to the TIFY family into the plant or enhancing the expression of the gene endogenous to the plant.

2. The method for conferring disease resistance to a plant according to claim 1, wherein the gene belonging to the TIFY family is selected from the group consisting of an OsTIFY11a gene, an OsTIFY11b gene, an OsTIFY11c gene, an OsTIFY11d gene, an OsTIFY11e gene, an OsTIFY11f gene, an OsTIFY11g gene, an OsTIFY10a gene, an OsTIFY10b gene, and an OsTIFY10c gene, and homologous genes thereof.

3. The method for conferring disease resistance to a plant according to claim 1, wherein the gene belonging to the TIFY family is selected from the group consisting of an OsTIFY11d gene, an OsTIFY11a gene, an OsTIFY11e gene, and an OsTIFY10a gene, and homologous genes thereof.

4. The method for conferring disease resistance to a plant according to claim 1, wherein the gene belonging to the TIFY family encodes any one of the following proteins (a) to (d):
(a) a protein comprising the amino acid sequence shown in SEQ ID NO: 2, 4, 6, 8, 10, 12, 14, 52, 54, or 56;
(b) a protein comprising an amino acid sequence derived from the amino acid sequence shown in SEQ ID NO: 2, 4, 6, 8, 10, 12, 14, 52, 54, or 56 by deletion, substitution, addition, or insertion of 1 or a plurality of amino acids, and having activity to confer disease resistance to a plant;
(c) a protein being encoded by a polynucleotide that hybridizes under stringent conditions to a polynucleotide consisting of a nucleotide sequence complementary to the nucleotide sequence shown in SEQ ID NO: 1, 3, 5, 7, 9, 11, 13, 51, 53, or 55, and having activity to confer disease resistance to a plant; and
(d) a protein comprising an amino acid sequence that has 70% or more identity to the amino acid sequence shown in SEQ ID NO: 2, 4, 6, 8, 10, 12, 14, 52, 54, or 56, and having activity to confer disease resistance to a plant.

5. The method for conferring disease resistance to a plant according to claim 1, wherein the gene belonging to the TIFY family is derived from a plant other than rice.

6. The method for conferring disease resistance to a plant according to claim 1, wherein the plant is a monocotyledon.

7. The method for conferring disease resistance to a plant according to claim 1, wherein the plant is rice.

8. A method for producing a plant body, comprising the steps of:
preparing a transgenic plant by introducing at least one of the genes belonging to the TIFY family into a plant or enhancing the expression of the gene endogenous to the plant; and
determining disease resistance of a progeny plant of the transgenic plant, so as to select a line with the significantly improved disease resistance.

9. The method for producing a plant body according to claim 8, wherein the gene belonging to the TIFY family is selected from the group consisting of an OsTIFY11a gene, an OsTIFY11b gene, an OsTIFY11c gene, an OsTIFY11d gene, an OsTIFY11e gene, an OsTIFY11f gene, an OsTIFY11g gene, an OsTIFY10a gene, an OsTIFY10b gene, and an OsTIFY10c gene, and homologous genes thereof.

10. The method for producing a plant body according to claim 8, wherein the gene belonging to the TIFY family is selected from the group consisting of an OsTIFY11d gene, an OsTIFY11a gene, an OsTIFY11c gene, and an OsTIFY10a gene, and homologous genes thereof.

11. The method for producing a plant body according to claim 8, wherein the gene belonging to the TIFY family encodes any one of the following proteins (a) to (d):
(a) a protein comprising the amino acid sequence shown in SEQ ID NO: 2, 4, 6, 8, 10, 12, 14, 52, 54, or 56;
(b) a protein comprising an amino acid sequence derived from the amino acid sequence shown in SEQ ID NO: 2, 4, 6, 8, 10, 12, 14, 52, 54, or 56 by deletion, substitution, addition, or insertion of 1 or a plurality of amino acids, and having activity to confer disease resistance to a plant;
(c) a protein being encoded by a polynucleotide that hybridizes under stringent conditions to a polynucleotide consisting of a nucleotide sequence complementary to the nucleotide sequence shown in SEQ ID NO: 1, 3, 5, 7, 9, 11, 13, 51, 53, or 55, and having activity to confer disease resistance to a plant; and
(d) a protein comprising an amino acid sequence that has 70% or more identity to the amino acid sequence shown in SEQ ID NO: 2, 4, 6, 8, 10, 12, 14, 52, 54, or 56, and having activity to confer disease resistance to a plant.

12. The method for producing a plant body according to claim 8, wherein the gene belonging to the TIFY family is derived from a plant other than rice.

13. The method for producing a plant body according to claim 8, wherein the plant is a monocotyledon.

14. The method for producing a plant body according to claim 8, wherein the plant is rice.
